SEQUENCE LISTING (19 sequences)

SEQ ID NO: 1 - protein, 11 aa, Bacteriophage T4
GLLRLNGDYVQ

SEQ ID NO: 2 - protein, 11 aa, Bacteriophage T4
GSNNVQFYADG

SEQ ID NO: 3 - protein, 16 aa, artificial
  Bacteriophage T4 with deletion
GLLRLNGDNVQFYADG

SEQ ID NO: 4 - protein, 21 aa, artificial
  Bacteriophage T4 with deletion and insert
GLLRLNGDGGGGGNVQFYADG

SEQ ID NO: 5 - protein, 38 aa, artificial
  Bacteriophage T4 with deletion and insert
GLLRLNGDGGGGARGVGKSALTIQLIGGGGNVQFYADG

SEQ ID NO: 6 - protein, 39 aa, artificial
  Bacteriophage T4 with delection and insert
GLLRLNGDGGGGEEYSAMRDQYMRTGEGGGGNVQFYADG

SEQ ID NO: 7 - DNA, 20 bp, artificial
  primer
ctattaacggacttttgaga

SEQ ID NO: 8 - DNA, 20 bp, artificial
  primer
ttcaatacgtccaatagttt

SEQ ID NO: 9 - DNA, 31 bp, artificial
  primer
gacgagctccttcgggttccctttttcttta

SEQ ID NO: 10 - DNA, 18 bp, artificial
  primer
ttgggtaactcgacatga

SEQ ID NO: 11 - DNA, 40 bp, artificial
  primer
ggcgatggtggcggtggcggcaatgtacaattttacgctg

SEQ ID NO: 12 - DNA, 39 bp, artificial
  primer
tacattgccgccaccgccaccatcgccatttaatctcaa

SEQ ID NO: 13 - DNA, 41 bp, artificial
  primer
ggcgatgagacggtaccgtctcaatgtacaattttacgctg

SEQ ID NO: 14 - DNA, 40 bp, artificial
  primer
tacattgagacggtaccgtctcatcgccatttaatctcaa

SEQ ID NO: 15 - DNA, 72 bp, artificial
  primer
gcgatggtggcggtggcgcccgcggcgtgggaaagagtgccctgaccatccagctgatcggtggcggtggca

SEQ ID NO: 16 - DNA, 72 bp, artificial
  primer
acattgccaccgccaccgatcagctggatggtcagggcactctttcccacgccgcgggcgccaccgccacca

SEQ ID NO: 17 - DNA, 75 bp, artificial
  primer
gcgatggtggcggtggcgaagaatactccgcaatgcgcgaccagtacatgcgcaccggtgaaggtggcggtggca

SEQ ID NO: 18 - DNA, 75 bp, artificial
  primer
acattgccaccgccaccttcaccggtgcgcatgtactggtcgcgcattgcggagtattcttcgccaccgccacca

SEQ ID NO: 19 - protein, 15 aa, artificial
  ras epitope
EEYSAMRDQVMRTGE

(19) United States
(12) Patent Application Publication
(10) Pub. No.: US 2008/0093219 A1
(43) Pub. Date: Apr. 24, 2008

(54) Magnetic Protein Nanosensors and Methods of Use

(75) Inventors: Edward B. Goldberg, Newton, MA (US); Robert P. Guertin, Boston, MA (US); Timothy P. Harrah, Cambridge, MA (US)

Correspondence Address: Marina Larson & Associates, LLC, P.O. BOX 4928, DILLON, CO, 80435, US

(73) Assignee: TUFTS UNIVERSITY, Boston, MA (US)

(21) Appl. No.: 11/854,200

(22) Filed: Sep. 12, 2007

Related U.S. Application Data
(63) Continuation-in-part of application No. PCT/US06/09487, filed on Mar. 15, 2006.
(60) Provisional application No. 60/825,766, filed on Sep. 15, 2006.
(60) Provisional application No. 60/662,563, filed on Mar. 15, 2005.

(51) Int. Cl. C07K 2/00; G01N 27/00; C12N 9/00

(52) U.S. Cl. 204/556; 530/402; 435/183; 977/796

(57) ABSTRACT

Nanometer-scale sensors useful in the detection of biological and chemical entities are formed from a protein rod body portion 41, a magnetic particle 42 affixed to the protein rod body portion 41; and an analyte binding moiety 43 disposed on the protein rod body portion 41 at a location remote from the magnetic particle 42, The analyte binding moiety specifically binds to the analyte 44 to form a sensor-analyte complex. The protein rod body portion may be formed from a tail fiber protein from a T even bacteriophage or a derivative thereof. Interaction of the analyte with the sensor will change the overall shape and size, and thus the ability of the sensor to move within a liquid sample in response to an applied magnetic field. Interaction can therefore be observed as a change in magnetic susceptibility or in relaxation time compared to that of a sensor in the absence of the analyte.

### STATEMENT OF RELATED APPLICATIONS

This application claims the benefit of U.S. provisional application No. 60/825,766, filed Sep. 15, 2006, and is a continuation-in-part of PCT Patent Application No. PCT/US2006/09487, filed Mar. 15, 2006, which application is incorporated herein by reference, which claim the benefit of U.S. Provisional Application No. 60/662,563 filed Mar. 15, 2005, all of which applications are incorporated herein by reference.

### STATEMENT REGARDING FUNDING

This invention was made with government support under grant A1057159 awarded by the National Institutes of Health. The US government may have certain rights in this invention.

### BACKGROUND OF THE INVENTION

This application related to a nanosensor that incorporates a magnetic particle on a protein rod for the detection of analytes in a liquid sample.

During the past decade, there has been increasing interest in the commercial and government sectors in the development of biosensors capable of selectively detecting specific biomolecules in a sample population. Examples of such populations range from cells and model organisms for pharmaceutical and genome research to samples of the environment for pathogens and biological-warfare-agent detection. In the case of genome research, it is necessary to discover gene sequences that provide a blueprint of the cell or organism through systematic identification of known and predicted genes. It is also of great interest to use so-called genomic arrays for gene expression monitoring and for screening for sequence variants or mutations. For these applications, it would be desirable to survey greater than 1 Mbit of genomic information on a single chip that is only a few cm² in area. In contrast, biosensors used to screen pathogens may have relaxed requirements on the number of different biomolecules that must be sensed simultaneously, while placing greater emphasis, e.g., on detection time, minimum detection levels, field durability, overall system size, energy requirement and cost.

The ability to design and produce very small molecular sensors (e.g., of nanometer dimensions) that can serve complex functions depends upon the use of appropriate materials that can be manipulated in predictable and reproducible ways, and that have the properties required for each novel application. Biological systems serve as a paradigm for sophisticated nanostructures. Living cells fabricate proteins and combine them into structures that are precisely formed and can resist damage in their normal environment. In some cases, intricate structures are created by a process of self-assembly, the instructions for which are built into the component polypeptides. Finally, proteins are subject to proofreading processes that insure a high degree of quality control. Therefore, there is a need in the art for methods and compositions that exploit these unique features of proteins to form constituents of synthetic nanosensors. The need is to design sensors whose properties can be tailored to suit the particular requirements of nanometer-scale technology.

Most biosensors are based on the selective recognition principles inherent in biological systems. Bioreceptors that have been used as sensing elements include biomolecules. Such as antibodies, enzymes, and nucleic acids. When a receptor undergoes a binding event with a target biomolecule, the information collected by the sensing element regarding the receptor-target attachment must be converted into a signal that can be easily measured. There are a number of transduction mechanisms that can be exploited for converting this attachment information, including optical, electrochemical, magnetic, and mass sensitive measurements. The choice of the particular bioreceptor/transducer combination will ultimately impact biosensor figures of merit, such as detection sensitivity, selectivity, repeatability, integrality, scalability, energy requirement and cost.

There are several techniques that rely on fluorescent tagging and/or optical readout. However, each of these techniques requires sophisticated optical detection, which may limit their utility. To overcome drawbacks of optical sensing, several groups have initiated research on biosensors that detect the presence of target biomolecules based on magnetic, electrical, or mass-sensitive transduction. For example, a bead-array-counter biosensor uses DNA-functionalized magnetic nanoparticles as the target probe and complementary DNA-covered magnetoresistive materials as the receptor/transducer (Baselt, D. R. et al. Biosensors & Bioeletronics 1998, 13, 731-739). When target and receptor DNA hybridize, the magnetic particles bind to the sensing element and modify the local magnetic field. This change is measured by monitoring the electrical resistance of the element, where the resistance is proportional to the number of hybridized beads on the element.

Recently, a method was proposed for the detection of biomolecules in an aqueous solution based on the detection of shifts in the frequency-dependence of the complex magnetic susceptibility of magnetic colloids due to an increase in hydrodynamic radius caused by specific binding with biomolecules (Connolly, J.; St. Pierre, T. G. Journal of Magnetism and Magnetic Materials 2001, 255, 156-160). A diagnostic sensor based on the same physical principles (i.e. the Brownian relaxation of magnetic nanoparticles suspended in liquids) was recently disclosed (Chung, S. H. et al. Appl. Phys. Letts. 2004, 85, 2971-2973). Chung et al. demonstrated that the characteristic time scale of the Brownian relaxation can be determined directly by alternating current susceptibility measurements as a function of frequency, as the peak in the imaginary part of the alternating current susceptibility shifts to lower frequencies upon binding a target molecule to a magnetic nanoparticle; this frequency shift is consistent with an increase in the hydrodynamic radius corresponding to the size of the target molecule.

Prieto-Astalan et al. have also used magnetic particles to study specific binding of prostate specific antigen to the surfaces of the bioparticles comprised of clusters of magnetic single domains of magnetite, which are coated with dextrin (Prieto-Astalan, A. P et al. Biosensors and Bioelectronics 2004, 19, 945-951; US 2003/9169032; US 2003/0076087; and WO 03/019188). Both groups showed that at sufficiently large amplitude of the magnetic field, or when using a dc-bias to the ac-excitation field, one observes a non-linear magnetic response. However, both of these approaches suffer from a number of limitations, e.g. low sensitivity.

### SUMMARY OF THE INVENTION

The present invention provides sensors, i.e. nanometer-scale sensors useful in the detection of biological and chemical entities. In accordance with one aspect of the invention, a sensor for detecting an analyte comprises

(a) a protein rod body portion;

(b) a magnetic particle affixed to the protein rod body portion; and

(c) an analyte-interacting moiety disposed on the protein rod body portion at a location remote from the magnetic particle, wherein the analyte interacting moiety interacts with the analyte to form a sensor-analyte product having different hydrodynamic properties that the sensor alone.

In some embodiments of the invention the analyte interacting moiety is an analyte binding moiety and the interaction of the sensor and the analyte is one that forms an analyte sensor complex, that is larger in size the sensor alone. In some embodiments, this analyte binding moiety specifically binds to the analyte. In other embodiments of the invention, the analyte interacting moiety is one which can be degraded by the analyte (for example a bound substrate for an enzyme) in which case the size of the sensor-analyte product will be less than that of the sensor alone.

The sensor analyte product may also be a product whose hydrodynamic properties differ in of the analyte not because of an actual change in the sensor itself, but because of change in the properties of a milieu in which the sensor is found. Thus, in one such case, the analyte interacting moiety is an enzyme and the analyte is a substrate for the enzyme, and the reaction of the enzyme and the substrate results in a change in the viscosity of the milieu which results in all observable change in the peak frequency detected for the sensor.

In specific embodiments, the protein rod body portion comprises a tail fiber protein from a T even bacteriophage or a derivative thereof, for example a tail fiber protein of T4 bacteriophage such as a gp 34, gp35, gp36 or gp 37 tail fiber protein or a derivative thereof. In other embodiments, the rod body portion comprises a viral fiber such as the T4 short tail fiber, or tail fiber proteins from adenovirus; and dimeric or trimeric coiled coils, including both naturally occurring and synthetic coiled coils. In addition, in some embodiments of the invention, antibodies or antibody fragments can serve the function of both the rod like protein and the analyte binding moiety since some antibody fragments achieve the desired rod like conformation. The protein rod body portion is a fusion protein comprising portions of two or more tail fiber proteins and/or a multimer of protein rod body portions, each having a magnetic nanoparticle and an analyte binding moiety associated therewith. The nature of this analyte binding moiety is not critical, provided that interaction of the analyte and the analyte binding moiety alters the hydrodynamic behavior of the sensor to a detectable extent.

The present invention also provides a method for detecting the presence of an analyte in a liquid sample comprising the steps of:

(a) placing a sensor in accordance with the invention into the liquid sample,

(b) applying an AC magnetic field to the sample containing the sensor, and

(c) observing the behavior of the sensor molecules in the magnetic field. Because binding of analyte to the sensor will change the overall shape and size, and thus the ability of the sensor to move within the liquid sample, binding can be observed as a change in magnetic susceptibility via a change in relaxation time compared to that of a sensor in the absence of the analyte.

### BRIEF DESCRIPTION OF THE FIGURES

FIGS. 1A and B depict a schematic representation of the T4 bacteriophage particle (FIG. 1A), and a schematic representation of the T4 bacteriophage tail-fiber (FIG. 1B).

FIGS. 2 A and B depicts a schematic representation of an electronic detection system. An amplitude and frequency-variable ac magnetic field is provided by the primary solenoid 201 wound outside two series-opposing secondary coils 202, 203. These are wound directly on a capillary 204 for close coupling to the solution within 205. When balanced, the output voltage of the pair of coils is null, unless media with unequal magnetic characteristics are introduced into one of the two secondaries, at which point the output voltage, detected by a phase-sensitive detector, is non-zero. The overall size of the system can be very small; secondaries wound with #50-wire are common. The primary coil, which is the source of the ac magnetic field, carries milli-ampere currents so is wound with a longer pitch and a somewhat more robust wire. The phase sensitive detector allows measurement of both the in-phase (real) component of the magnetic susceptibility, χ′ (ω), and the out-of-phase (imaginary) component, χ″ (ω). Key components are the ac current generator, null electronic circuitry for balancing secondaries in absence of unequal media (N), phase sensitive detector (φ), α is the sample capillary with media exposed to target protein, virus, cell or other molecule, and β is the sample capillary with media unexposed.

FIGS. 3A-D depict attachment to a phage by a monoclonal antibody. (A) Treatment of phage with mAb and secondary anti-serum. Each phage type was treated with 1 μg of mAb as described in Example 7. (B) Time course of mAb treatment. SΔ1ras2 phage were treated with 3 μg of mAb for the indicated time before a 30 min incubation with secondary anti-serum. (C) Dose-response study of SΔ1ras2 phage with varying amounts of mAb. (D) Effect of treating mAb with free epitope before inactivation of SΔ1ras2.

FIG. 4 depicts a schematic of a nanosensor. The construct is an engineered viral protein 41 conjugated to a single domain nanoparticle 42. The distal region of the rod-like protein displays peptide/protein ligands 43 that bind to targets in solution. Binding is detected via resultant changes in rotational diffusion time by measuring the loss term of the complex magnetic susceptibility.

### DETAILED DESCRIPTION OF THE INVENTION

### 1. Overview

The present invention related to molecular protein/magnetic particle composite sensors that have affinity for a selected target analyte. In one embodiment, the protein component of the sensor is a rigid, rod-like structure engineered from a T4 tail-fiber gene. Predetermined loci for binding nano-functional moieties can be genetically engineered along the distal half of the T4 tail-fiber (a structure that is about 2 nm wide by about 70 nm long—i.e., a “strut”). We have engineered a specific 23 amino acid ras epitope display in a manner that makes it available to the surrounding medium. This enables its target, a monoclonal ras antibody, to bind tightly to the strut in solution. The generalization is that target specific binding moieties can serve to capture targets (e.g. molecules, viruses or cells) in solution. See Hyman, P. et al. PNAS 2000, 99, 4888-4893. By attaching, in addition, a magnetic particle (for example a nanoparticle with a diameter of about 10 nm ii) to the strut in solution, we have a sensor.

The sensor of the present invention provides designability and rigid aspect ratio. When exposed to a weak ac magnetic field spectrum, there will be a peak of the imaginary component of the magnetic susceptibility, χ″, at a frequency determined mainly by Brownian motion of the sensor. The Brownian motion of such rigid asymmetric sensors is a cubic function of the length of the strut. On this basis sensors of different lengths and different target analyte specificity can be synthesized whose peak frequencies will be significantly separated in the spectrum and facilitate de-convolution.

One aspect of the present invention pertains to nano-sensors, i.e., nanometer-sized sensors useful in the detection of biological and chemical entities. The basis for the sensors described herein is a multivalent “nano-strut” (i.e., a long and thin rigid rod) composed of T-even tail-fiber proteins and variants thereof. In a preferred embodiment, the present invention pertains to a nano-strut engineered to bind a single domain nanoparticle, or other strongly magnetic molecular entity, and a target entity.

Tail fiber proteins used in the sensors of the invention can be modified in various ways to form novel rod structures with different properties. Specific internal peptide sequences can be deleted without affecting their ability to form trimers and associate with their natural tail-fiber partners. Alternatively, they can be modified so that they contain additional functional groups which enable them to interact with heterologous binding moieties. The present invention also encompasses fusion proteins that contain sequences from two or more different tail-fiber proteins. In another aspect, the present invention provides nano-sensors comprising native and modified tail-fiber proteins of bacteriophage T4.

The surface display ligands (for example peptide display ligands “PDL”) of the strut are genetically engineered into sections (e.g., the display ligand will bind a single domain (superparamagnetic or paramagnetic) nanoparticle, and at a separate remote location, for example at the other end of the protein rod, will be a display specific for the selected target/analyte).

We have shown that a nano-strut composed of T4 tail-fiber proteins is a homo-trimer, and therefore most likely has three-fold symmetry along the long axis. This implies that any display is repeated three times about the specific locus on the axis. When the strut is put into a solution containing target(s), e.g., in a capillary, the struts will orient to some degree and point in the long direction. This orientation can be improved by a homogeneous DC magnetic field. By imposing a magnetic field gradient, the struts increase their concentration. In an ac magnetic field, at certain resonance frequencies, the struts will respond by an oscillatory movement, either along the axis or at an angle to it. The frequency and direction will depend on the overall size and the mass distribution of the strut and its dependants. Thus, struts with a bound target can be distinguished from those without bound targets and quantified. Alignment of the struts and their dynamics may be monitored by passage of polarized light perpendicular to the tube axis (when using a DC field).

By utilizing nano-struts of different lengths, each genetically engineered to attract a different target, we can simultaneously assay several targets. Since the resonance frequency and strength are a function of length, mass and shape, the targets can be recognized in the same test by scanning the proper frequencies. Importantly, the test can be confirmed by titration using first a specific free peptide competitor and then a second. These peptides are same sequence as the target recognition peptides engineered into the struts and function as competitive inhibitors. If added one at a time, they should diminish each resonance amplitude in a specific manner. For example, a target molecule with two fold symmetry may well have two target sites and therefore the inhibition curve will be different from that of a target molecule which has only one. In each case (at each resonance frequency) the final value will give the same value as the starting value (before the addition of any target molecules). In this way the specificity of the signal can be checked (based on previous control data) and the baseline of the electronics and optics checked for changes or errors in the hardware. Finally, this approach allows for automated as well as manual analysis for specific sets of targets in a single sample.

### 2. Definitions

For convenience, before further description of the present invention, definitions of certain terms employed in the specification, examples, and appended claims are collected here.

“AC” and “ac” refer to alternating current.

“DC” and “dc” refer to direct current.

“Chimers” are defined herein as chimeric proteins in which at least the amino- and carboxy-terminal regions are derived from different original polypeptides, whether the original polypeptides are naturally occurring or have been modified by mutagenesis. The peptide portion of a chimer is denoted by a tilda, “˜”, and can be designated, e.g., gp(37˜36) for a monomer and P(37˜36) for a trimer.

The designation “cp” denotes a monomeric polypeptide, while the designation “P” denotes homooligomers; for example, P34, P36, and P37 are homotrimers. “Homotrimers” are defined herein as assemblies of three substantially identical protein subunits that form a defined three-dimensional structure.

An isolated polypeptide that “consists essentially of” a specified amino acid sequence is defined herein as a polypeptide having the specified sequence or a polypeptide that contains conservative substitutions within that sequence. Conservative substitutions, as those of ordinary skill in the art would understand, are ones in which an acidic residue is replaced by an acidic residue, a basic residue by a basic residue, or a hydrophobic residue by a hydrophobic residue. Also encompassed is a polypeptide that lacks one or more amino acids at either the amino terminus or carboxy terminus, up to a total of five at either terminus, when the absence of the particular residues has no discernable effect on the structure or the function of the polypeptide in practicing the present invention.

As used herein the prefix “nano” indicates a structure of small size measured in nanometers, but generally not greater than 1000 nm (1 μm) in any dimension. As a practical matter, however, the size of the sensors of the invention is substantially dependent on the size of the analyte, since greater observable change in the properties of the sensor in the magnetic field will occur with a greater percentage size increase when the sensor analyte complex is formed. Thus, smaller analytes are most effectively detected with smaller sensors, while larger analytes can be detected with larger sensors.

As used herein, the term “specific binding” will be understood in the manner conventional in the art to refer to non-covalent interactions such as those formed between antigens and their cognate antibodies, bio-receptors and their cognate ligands and like pairings, as may be reflected in binding, displacement or competition assays.

The term “interacts” or “interaction” refers to the formation of an association between the analyte binding moiety and an analyte provided they result in a measurable difference in the hydrodynamic properties, or average hydrodynamic properties of the sensor-analyte complex, as compared to the sensor alone. The term “interaction” includes specific binding, but may also include more transient and less specific interactions such as the interaction of coiled coils or viral adhesins with polysaccharides or non-sequence specific interactions with nucleic acids (protein-DNA, protein-RNA, intercalating agent-DNA and the like.)

In the sensor of the invention, the position of the analyte binding is “remote” from the magnetic particle. In some embodiments, the magnetic particle and the analyte binding moiety are disposed at or near opposing ends of the protein rod body portion to maximize the change in shape and size when a sensor/analyte complex is formed. This degree of separation is not required, however, and the term “remote” is intended to reflect merely that the analyte binding moiety and the location of the magnetic particle are sufficiently separated that the there is no interference in binding both analyte and magnetic particle.

### 3. Principles of Diagnostic Sensors of the Present Invention

Two labs have reported initial feasibility data on biosensors based on magnetic susceptibility measurements of the Brownian relaxation of spherical particles (Chung, S. H. et al. Appl. Phys. Letts. 2004, 85, 2971-2973; Prieto-Astalan, A. P et al. Biosensors and Bioelectronics 2004, 19, 945-951; US 2003/9169032; US 2003/0076087; and WO 03/019188). The technique relies on a shift in the peak frequency of the imaginary (loss) component of the complex ac magnetic susceptibility, χ″(ω), first suggested as a biosensor by Connolly and St. Pierre (Connolly, J.; St. Pierre, T. G. Journal of Magnetism and Magnetic Materials 2001, 255, 156-160). The peak amplitude of χ″(ω) is shifted to a lower frequency when magnetic particles in solution bind target molecules, increasing the effective hydrodynamic radius and, thus, the Brownian rotational diffusion time. For particle radii of approximately 25-350 nm, a change in susceptibility can be directly measured using relatively simple equipment via changes in the inductance of a sample pickup coil in the presence of a small external ac excitation magnetic field. At larger radii, peaks in χ″(ω) may disappear altogether, providing indirect detection of even larger analytes.

The imaginary component of complex magnetic susceptibility, χ″(χ), is described by the following relation:
χ″(ω)=χₒωτ/[1+ωτ²] Eq. 1
where χₒ is the inherent susceptibility, ω is the frequency, and τ is the relaxation time. For blocked superparamagnetic particles in an ac magnetic field, this relaxation time is primarily dependant on Brownian motion. Thus, for spherical particles, τ can be approximated by the Brownian rotational diffusion time (τᵣ):
τ_{r,sphere}=4πηr³/kT [Eq.2]
and for rods (Kirkwood, J. G. et al. J. Chem. Phys. 1951, 19, 281-283):
τ_{r,rod}=πηL³/3 kT(ln(L/d−0.8)) [Eq.3]
where η is the fluid viscosity, r the spherical particle radius, L the rod length, d the rod diameter, k is Boltzmann's constant and T the absolute temperature.

Using equation 1, it can be shown that χ″ is maximum at ωτ=1, or, put another way, ωₘₐₓ=1/τᵣ. Since τᵣ is a function of r³ or L³ depending on sensor geometry, this means that upon binding to an analyte(s), the peak imaginary susceptibility will decrease proportional to the difference in effective sensor hydrodynamic size(s). Since this proportionality is cubed, the theoretical resolution of the method is quite high. However, for spherical particles, at diameters of 300-350 nm, relaxation times become so large that observation becomes impractical. We believe that rod-like particles overcome this limitation, offering extended range as well as increased resolution across all frequencies. For example, using equations 2 and 3 to compare a 25 nm radius sphere to a 50 nm long rod with a L/d ratio of 20, we estimate that the rod will have approximately 5 times the peak frequency (extending the size range of target molecules and moving to a range of higher instrument sensitivity) with twice the resolution (frequency separation of χ″ peaks based on a changed in τ equal to an effective 1 nm reduction in length/radius).

In addition, biological production of the protein based rod-like segment of our sensor offers the opportunity to engineer multiple binding motifs at discrete locations along the length of a sensor, as well as allowing us to vary easily the overall length of the sensor with nearly monodisperse molecular weight distributions. The capability to readily modify the sensor construct, altering both its binding specificity and hydrodynamic behavior creates a large variable space of available combinations in which to optimize the sensing of single and multiple analytes under a variety of conditions. Monodispersity of protein based sensors should also result in peak sharpening (Connolly, J. and St. Pierre, T. G., J. Mag. And Mag. Matl, 2001, 255, 156-160).

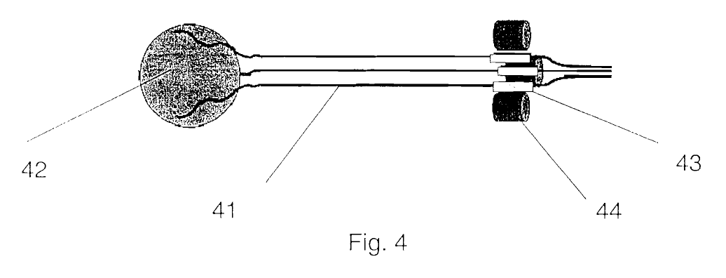

One embodiment of our sensor (FIG. 4) is based on the distal tail-fiber of bacteriophage T4. This is a rigid rod-like trimerie protein 41 approximately 2.5×50 nm. The sensor is created by the conjugation of a magnetic nanoparticle 42 at one end of the protein 41 and the engineering of a peptide/protein based binding motif(s) 43 at another point(s) along the rod. The binding motifs 43 bind to analyte 44. We have been successful in engineering tail-fiber variants of different lengths. These could display peptide motifs accessible for targeted antibody binding. These variants still produce infectious bacteriophage, and, as a result, are easily produced at high yield in Escherichia coli. Avidin/biotin conjugation is anticipated to provide magnetic nanoparticle attachment (N-terminal covalent attachment chemistry and phage display based discovery of peptides for direct nanoparticle binding are considered alternatives). We have already developed display peptides on the surface of the tail-fiber that bind antibody. Assay of anthrax infection can be done using a high affinity display peptide specific to Bacillus anthracis protective antigen protein (PA). PA is the central soluble component of the tripartite anthrax toxin in blood and is most often used as an index of toxin production. With a different affinity display we can detect anthrax spores. (Collier, R. J.; Young, J. A. T. “Anthrax toxin” Ann Rev Cell & Dev Biol 2003, 19, 45-70.)

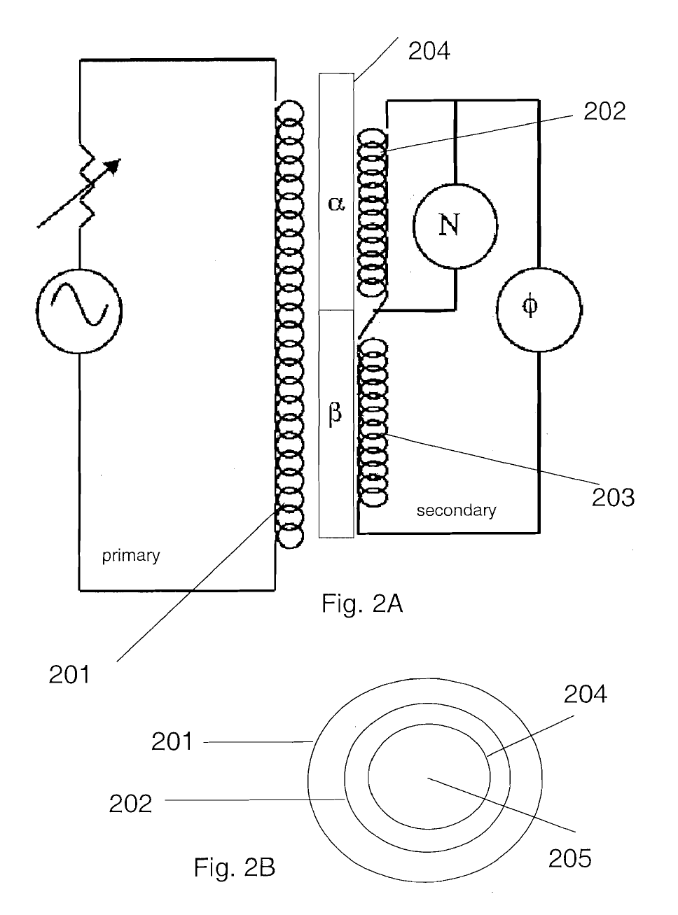

The sensitivity of the sensors of the present invention if confirmed through computer modeling. Simulation of the hydrodynamic properties of tail-fiber based sensors was conducted using HYDRO (Garcia de la Torre, et al, Biophysical Journal 1994, 67: 530-531). HYDRO is based on the work of Bloomfield and colleagues (see Carrasco, et al., Biophysical Journal 1999, 75: 3044-3057 for a review) and uses bead modeling to approximate the shape of complex macromolecules. Based on available microscopy data (Hyman, et al., 2002 and Cerritelli, et al., 1996), the T4 37Δ1 distal half fibers (DHFs) useful in sensors of the invention have dimensions approximately 50 nm×2.5 nm and were modeled as 20 beads with a diameter of 2.511111. The Mag/Rod˜sensor was modeled as the DHF with a 5 nm magnetic particle appended to the end of the rod. Three additional cases were considered: (1) the sensor plus 3 bound Bacillus anthracis protective antigen (PA) molecules (modeled as cylinders 3.5 nm×10 nm from crystallographic data of Pesota, et al., 1997) arranged uniformly around the terminus opposite the magnet (FIG. 2); (2) the sensor plus a single bound antibody (modeled after IgG3 in Garcia de la Torre, 2001 and extending in the long axis of the rod); and (3) the sensor plus a B. anthracis spore (approximated as a sphere with D=3000 nm). Mean relaxation times (τᵣ) were calculated for each case and are shown in Table 2 along with the % Change in relaxation time on target binding and the anticipated peak frequency (ω).

TABLE 2

| | | | ω = l/τᵣ |
|---|---|---|---|
| Tail Fiber | τᵣ (s) | % Change | (kHz) |
| Distal Half Fiber | 1.168E−07 | | 1,363 |
| DHF˜Sensor | 1.635E−07 | | 973 |
| + PA | 5.658E−07 | 246 | 281 |
| + MAb | 4.713E−06 | 2783 | 34 |
| + Spore | 3.507E+00 | 2.14E+09 | 0 |

These simulations demonstrate that the use of a rigid rod-like nanosensor in this scheme is an advantageous approach. Model data predicts significant, differentiable shifts in peak frequency for both MAb and PA binding. In addition, the large target size range predicted earlier is readily apparent in Table 2, where good peak separation is predicted at measurable frequencies (5 Hz-13 MHz, Connolly and St. Pierre, 2001) across a 20 fold increase in analyte size. As efficient multiplex detection (i.e. using a single magnetic particle) depends on the ability to detect the addition of multiple particles, we believe that this extended range is valuable. Reduction of peak frequency essentially to zero upon spore binding, resulting in a loss of detectable signal is also predicted as expected. Optimization of differences in frequency or relaxation time can also be achieved through modification of buffer viscosity and/or assay temperature.

In order to expedite measurement of the frequency dependence of the magnetic susceptibility, a tailored current pulse may be applied to the primary coil of the primary/secondary coil set. The tailored pulse has frequency components that span the necessary range of frequencies that are required for the measurement. This differs from a “white noise” approach in the US Patent Publication 2003/0169032, which, in effect, calls for a much larger (essentially infinite) frequency response than is necessary for the measurement.

For a given un-tagged rod/magnetic nanoparticle complex, the frequency range spanning the maximum in the complex part of the magnetic susceptibility can be predetermined in order to construct the pulse. Repetitive pulses and the use of signal averaging techniques build up statistics to determine the response of the TF/magnetic nanoparticle complex to outside agents.

### 4. T4 Bacteriophage Tail-Fibers

Although the invention is principally described in terms of bacteriophage T4 tail-fiber proteins, it will be understood that the invention is also applicable to tail-fiber proteins of other T-even-like phage, such as the tail-fiber proteins of the T4 (e.g., T4, Tula, Tulb, etc.) or the T2 (e.g., T2, T6, K3, Ox2, M1, etc.) families.

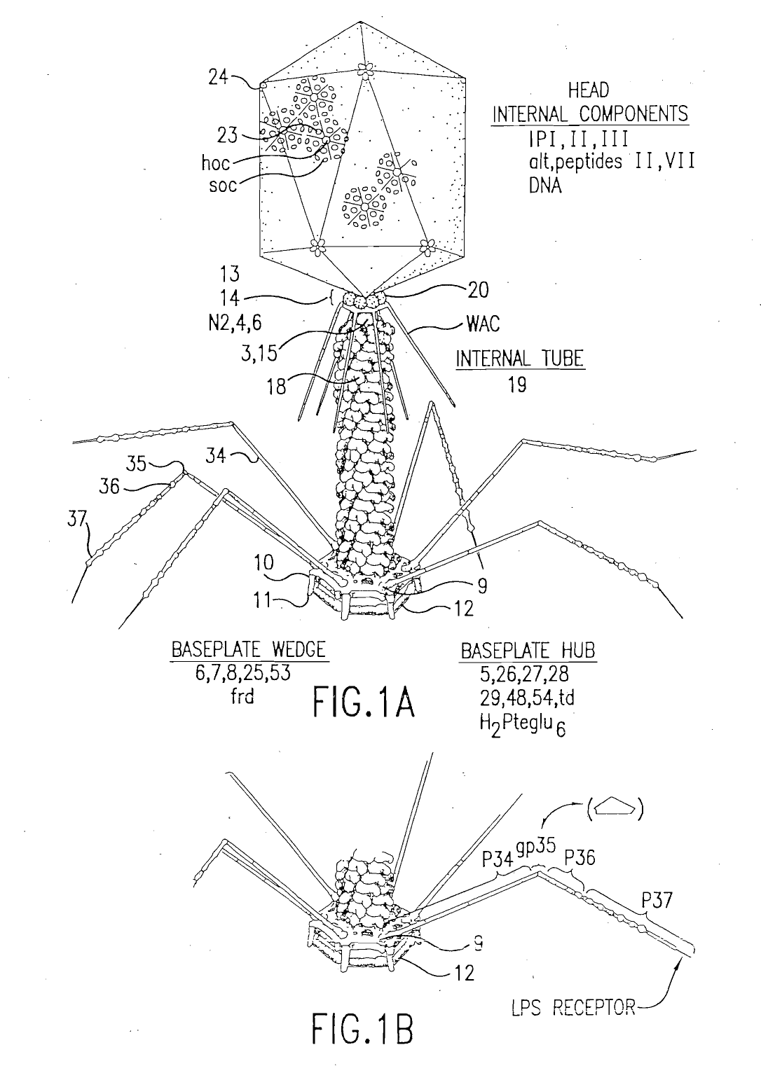

Bacteriophage (phage) T4 is one of the archetypal members of the family Myoviridae or T-even-like phage. These viruses are characterized by a large, elongated icosohedral head (which contains the phage DNA), a contractile tail sheath (and a “morphing” baseplate to stabilize the phage perpendicular to the cell, and to penetrate the outer cell wall preparatory to DNA injection), and tail-fibers (which contain the reversible receptors phage of the host receptors and trigger infection) (Wood, W. B. (1979) Harvey Lect. 73, 203-223; and Eiserling, F. A. & Black, L. W. (1994) in Molecular Biology of Bacteriophage T4, ed. Karam, J. D. (Am. Soc. Microbiol. Press, Washington, D.C.), pp. 209-212; FIG. 1A). The tail-fiber proteins have an unusual quaternary structure of long, thin (3 nm×150 nm), rigid rods (Beckendorf, S. K. J. Mol. Biol. 1973, 73, 37-53). Their function is to transduce chemical recognition of the bacteria host into a mechanical force on the phage base plate, essentially acting as a set of cooperative levers. This mechanical stress triggers a series of protein conformational changes that lead to entry of the phage DNA into the cell (Arscott, P. G.; Goldberg, E. B. Virology 1976, 69, 15-22; and Crawford, J. T.; Goldberg, E. B. J. Mol. Biol. 1980, 139, 679-690).

The three main tail-fiber proteins, P34, P36 and P37 (Note: gpX (gene product) refers to the monomeric product of gene X, whereas PX refers to the matured, multimeric complex of gpXs that has assembled into the structure that is found in the phage T4 virion) are principally composed of trimeric parallel ″-helical rods (Earnshaw, W. C.; Goldberg, E. B.; Crowther, R. A. J. Mol. Biol. 1979, 132, 101-131). The monomer, gp35, that forms the angle in the tail-fiber, probably has a more complex structure. The joints between the homotrimeric segments are also likely to have a more complex structure, but there is no evidence that the central rod regions have any tertiary structure (i.e., interactions between distant amino acid residues; Beckendorf, S. K. J. Mol. Biol. 1973, 73, 37-53). The extended parallel β-helical secondary structure should directly support the rigid rod quaternary structure. We have shown that deletions or additions to the central rod regions which maintain the β-helical structure should permit alteration in tail-fiber length without greatly affecting overall structural integrity. Further, the binding domains at the ends of the proteins should form separate functional domains from the central, rigid rod domain. Finally, the β-helical structure should contain turns and loops that can be expanded with functional peptides without disrupting the quaternary structure.

In one embodiment, the present invention pertains to a class of protein building blocks whose dimensions are measured in nanometers, which are useful in the construction of sensors. It is believed that the basic unit of these “building blocks” is a homotrimer composed of three identical protein subunits having a helical-″ configuration, although other oligomeric structures are possible. Thus, as will be apparent, references to a “homotrimer” or “trimerization” as used herein will in many instances be construed as also referring to other oligomers or oligomerizations. These long, stiff, and stable rod-shaped units can assemble with other rods using coupling devices that can be attached genetically or in vitro. The ends of one rod may attach to different ends of other rods or similar rods. Variations in the length of the rods, in the angles of attachment, and in their flexibility characteristics permit differently-shaped structures to self-assemble in situ. In this manner, the units can self-assemble into predetermined larger structures. The self-assembly can be staged to form structures of precise dimensions and uniform strength due to the relatively flawless biological manufacture of the components. The rods can also be modified by genetic and chemical modifications to form predetermined specific attachment sites for other chemical and biological entities.

An important aspect of the present invention is that the protein units can be designed so that they comprise rods of different lengths, and can be further modified to include features that alter their surface properties in predetermined ways and/or influence their ability to join with other identical or different units. Further, the self-assembly capabilities can be expanded by producing chimeric proteins that combine the properties of two different members of this class. This design feature may be achieved by manipulating the structure of the genes encoding these proteins.

As detailed below, the compositions and methods of the present invention take advantage of the properties of the natural proteins, i.e., the resulting structures are stiff, strong, stable in aqueous media, heat resistant, protease resistant, and can be rendered biodegradable. A large quantity of units can be fabricated easily in microorganisms. Further, for case of automation, large quantities of parts and subassemblies can be stored and used as needed. In a preferred embodiment, the sequences of the protein subunits of the instant invention are based on the components of the tail-fiber of the T4 bacteriophage of E. coli. It will be understood that the principles and techniques can be applied to the tail-fibers of other T-even-like phages, or other related bacteriophages that have similar tail and/or fiber structures.

The structure of the T4 bacteriophage tail-fiber (illustrated in FIG. 1) can be represented schematically as follows: (N=amino terminus, C=carboxy terminus):

N[P34]C-[gp35]-N[P36]C—N[P37]C.

P34, P36, and P37 are all stiff, rod-shaped protein homotrimers in which three identical ″β-sheets, oriented in the same direction, are fused face-to-face, by hydrophobic interactions between, juxtaposed with a 1200 rotational axis of symmetry through the long axis of the rod. Gp35, by contrast, is a monomeric polypeptide that attaches specifically to the N-terminus of P36 and then to the C-terminus of P34 and forms an angle joint between two rods.

During T4 infection of E. coli, three gp37 monomers trimerize to form a P37 homotrimer; the process of trimerization is believed to initiate near the C-terminus of P37 and to require two E. coli chaperone proteins. (A variant gp37 with a temperature sensitive mutation near the C-terminus, which only requires only one chaperone, gp57, for trimerization, may also be used in the instant invention.) Once trimerized, the N-terminus of P37 initiates the trimerization of three so that each layer of 3 identical interacting ″-strands, form a 3-fold symmetry axis. The “turns or loops” between these layers facilitate registration of these layers. The joint between the C-terminus of P36 and the N-terminus of P37 is tight and stiff but non-covalent. The terminus of P36 then attaches to a gp35 monomer; this interaction stabilizes P36 and forms the elbow of the tail-fiber. Finally, the gp35 monomer joins the P36-P37 rod to the P34 rod (that also needs the gp57 chaperone for trimerization) at a fixed angle. Thus, self assembly of the tail-fiber is regulated by a predetermined order of interaction of specific subunits whereby structural maturation caused by formation of the first subassembly permits interaction with new (previously disallowed) subunits. This results in the production of a structure of exact specifications from a random mixture of the components.

In accordance with the present invention, the genes encoding these proteins may be modified so as to make rods of different lengths with different combinations of ends. The helix properties of the native proteins are particularly advantageous in this regard. First, the β-helix is composed of parallel triangular groups of β-strands thought to form a prism shape with β-bends or loops at the three long edges of the prism. Second, the amino acid side chains of the strands alternate up and down out of the plane of the layer formed by the three homotrimeric strands. The first property allows loops to be extended to form symmetric and specific attachment sites between the L and R surfaces, as well as to form attachment sites for other structures. In addition, the core sections of the O-sheet can be shortened or lengthened by genetic manipulations, e.g., by splicing DNA regions encoding ″β-loops, on the same edge of the sheet, to form new loops that exclude intervening peptides, or by inserting segments of peptide in an analogous manner by splicing at bend angles. The second property allows amino acid side chains extending above and below the surface of the ″β-sheet to be modified by genetic substitution or chemical coupling. Importantly, all of the above modifications are achieved without compromising the structural integrity of the rod. It will be understood by one skilled in the art that these properties allow a great deal of flexibility in designing units that can assemble into a broad variety of structures, some of which are detailed below.

### 5. Structural Units

The rods of the present invention function like “struts”, e.g., wooden dowels, 2×4 studs or steel beams used in construction. In this case, the surfaces are exactly reproducible at the molecular level and thereby fitted for specific attachments to similar or different units rods at fixed joining sites. The surfaces are also modified to be more or less hydrophilic, including positively or negatively charged groups, and have protrusions built in for specific binding to other units or to an intermediate joint with two receptor sites. The three dimensions of the rod are defined as: x, for the back (B) to front (F) dimension; y, for the down (D) to up (U) dimension; and z, for the left (L) to right (R) dimension.

One dimensional multi-unit rods can be most readily assembled from single unit rods joined along the x axis but regular joining of subunits in either of the other two dimensions will also form a long structure, but with different cross sections than in the x dimension.

### 6. Design and Production of the Rod Proteins

In certain embodiments, the protein subunits that are used to construct the nanosensors of the present invention are based on the four polypeptides that comprise the tail-fibers of bacteriophage T4, i.e., gp34, gp35, gp36 and gp37. The genes encoding these proteins have been cloned, and their DNA and protein sequences have been determined (for gene 36 and 37 see Oliver et al. J. Mol. Biol. 1981, 153, 545-568). The DNA and amino acid sequences of genes 34, 35, 36 and 37 are set forth in U.S. Pat. No. 5,877,279, which is hereby incorporated by reference in its entirety.

Gp34, gp35, gp36, and gp37 are produced naturally following infection of E. coli cells by intact T4 phage particles. Following synthesis in the cytoplasm of the bacterial cell, the gp34, 36, and 37 monomers form homotrimers, which are competent for assembly into maturing phage particles. Thus, E. coli serves as an efficient and convenient factory for synthesis and trimerization of the protein subunits described herein below. In practicing the present invention, the genes encoding the proteins of interest (native, modified, or recombined) are incorporated into DNA expression vectors that are well known in the art, as discussed below. These circular plasmids typically contain selectable marker genes (usually conferring antibiotic resistance to transformed bacteria), sequences that allow replication of the plasmid to high copy number in E. coli, and a multiple cloning site immediately downstream of an inducible promoter and ribosome binding site. Examples of commercially available vectors suitable for use in the present invention include the pET system (Novagen, Inc., Madison, Wis.) and Superlinker vectors pSE280 and pSE380 (Invitrogen, San Diego, Calif.).

In certain embodiments, the protocol is to 1) construct the gene of interest and clone it into the multiple cloning site; 2) transform E. coli cells with the recombinant plasmid; 3) induce the expression of the cloned gene; 4) test for synthesis of the protein product; and 5) test for the formation of functional homotrimers. In some cases, additional genes are also cloned into the same plasmid, when their function is required for trimerization of the protein of interest. For example, when wild-type or modified versions of gp37 are expressed, the bacterial chaperon gene 57 may also be included; when wild-type or modified gp36 is expressed, the wild-type version or a modified version of the gp37 gene may be included. The modified gp37 should have the capacity to trimerize and contain an N-terminus that can chaperon the trimerization of gp36. This method allows the formation of monomeric gene products and, in some cases, maturation of monomers to homotrimeric rods in the absence of other phage-induced proteins normally present in a T4-infected cell.

Steps 1-4 of the aforementioned protocol may be achieved by methods that are well known in the art of recombinant DNA technology and protein expression in bacteria. A representative of this type of chimer, the fusion of gp37-36, is described in Example 2. The preferred hosts for production of these proteins (Step 2) are E. coli strain BL21(DE3) and BL21(DE3/pLysS) (available commercially from Novagen, Madison, Wis.), although other compatible recA strains, such as HMS174(DE3) and HMS174(DE3/pLysS) can be used. Transformation with the recombinant plasmid (Step 2) may be accomplished by standard methods (Sambrook, J., Molecular cloning, Cold Spring Harbor Laboratories, Cold Spring Harbor, N.Y.; this monograph is also the source for many standard recombinant DNA methods used in this invention.)

Transformed bacteria may be selected by virtue of their resistance to antibiotics, e.g., ampicillin or kanamycin. The method by which expression of the cloned tail-fiber genes is induced (Step 3) depends upon the particular promoter used. A preferred promoter is plac (with a lac I^{q} on the vector to reduce background expression), which can be regulated by the addition of isopropylthiogalactoside (IPTG). A second preferred promoter is pT7#10, which is specific to T7 RNA polymerase and is not recognized by E. coli RNA polymerase. T7 RNA polymerase, which is resistant to rifamycin, is encoded on the defective lambda DE lysogen in the E. coli BL21 chromosome. T7 polymerase in BL21 (DE3) is super-repressed by the laciq gene in the plasmid and is induced and regulated by IPTG.

Typically, a culture of transformed bacteria is incubated with the inducer for a period of hours, dulling which the synthesis of the protein of interest is monitored. In the present instance, extracts of the bacterial cells are prepared, and the T4 tail-fiber proteins are detected, for example, by SDS-polyacrylamide gel electrophoresis. Once the modified protein is detected in bacterial extracts, it is usually necessary to ascertain whether or not it forms appropriate homotrimers (Step 4). This may be accomplished initially by testing whether the protein is recognized by an antiserum specific to the mature trimerized form of the protein. Resistance to protease degradation is also a useful assay for native structure (Granboulan, P, J. Gen. Micro. 1983, 129, 2217-2228).

Tail-fiber-specific antisera may be prepared as described (Edgar, R. S; Lielausis, I. Genetics 1965, 52, 1187-1200; and Ward, S. et al. J. Mol. Biol. 1970, 54, 15-31). Briefly, whole T4 phage may be used as an immunogen; optionally, the resulting antiserum is then adsorbed with tail-less phage particles, thus removing all antibodies except those directed against the tail-fiber proteins. In a subsequent step, different aliquots of the antiserum may be adsorbed individually with extracts that each lack a particular tail-fiber protein. For example, if an extract containing only tail-fiber components P34, gp35, and gp36 (derived from a cell infected with a mutant T4 lacking a functional gp37 gene) is used for absorption, the resulting antiserum will recognize only mature P37 and trimerized P36-P37. A similar approach may be used to prepare individual antisera that recognize only mature (i.e., homotrimerized) P34 and P36 by adsorbing with extracts containing distal half tail-fibers or P34, gp35 and P37, respectively, and gp monomers of all. An alternative is to raise antibody against purified tail-fiber halves, e.g., P34 and gp35-P36-P37. Anti gp35-P36-P37 can then be adsorbed with P36-P37 to produce anti-gp35, and anti-P36 can be produced by adsorption with P37 and gp35. Anti-P37, anti-gp35, and anti-P34 can also be produced directly by using purified P37, gp35, and P34 as immunogens. Another approach is to raise specific monoclonal antibodies against the different tail-fiber components or segments thereof.

Specific antibodies to subunits or tail parts may be used in any of the following ways to detect appropriately homotrimerized tail-fiber proteins: 1) Bacterial colonies may be screened for those expressing mature tail-fiber proteins by directly transferring the colonies, or samples of lysed or unlysed cultures, to nitrocellulose filters, lysing the bacterial cells on the filter if necessary, and incubating with specific antibodies. Formation of immune complexes may then be detected by methods widely used in the art (e.g., secondary antibody conjugated to a chromogenic enzyme or radiolabelled Staphylococcal Protein A). This method is particularly useful to screen large numbers of colonies e.g., those produced by EXO-SIZE deletion as described above. 2) Bacterial cells expressing the protein of interest may be first metabolically labeled with ³⁵S-methionine, followed by preparation of extracts and incubation with the antiserum. The immune complexes may then be recovered by incubation with immobilized Protein A followed by centrifugation, after which they may be resolved by SDS-polyacrylamide gel electrophoresis.

In specific embodiments, the chimers of the invention comprise at least about the first 50 (N-terminal) amino acids of a first tail-fiber protein fused via a peptide bond to at least about the last 50 (C-terminal) amino acids of a second tail-fiber protein. The first and second tail-fiber proteins can be the same or different proteins. In another embodiment, the chimers comprise an amino acid portion in the range of the first 10-60 amino acids from a tail-fiber protein fused to an amino acid portion in the range of the last 10-60 amino acids from a second tail-fiber protein. In another embodiment, each amino acid portion is at least 20 amino acids of the tail-fiber protein. The chimers comprise portions, i.e., not full-length tail-fiber proteins, fused to one another. In a preferred embodiment, the first tail-fiber protein portion of the chimer is from gp37, and the second tail-fiber protein portion is from gp36. Such a chimer (gp37-36 chimer), after oligomerization to form P37-36, can polymerize to other identical oligomers. A gp36-34 chimer, after oligomerization to form P36-34, can bind to gp35, and this unit can then polymerize. In another embodiment, the first portion is from gp37, and the second portion is from gp34. In a preferred embodiment, the chimers of the invention are made by insertions or deletions within a β turn of the β structure of the tail-fiber proteins. Preferably, insertions into a tail-fiber sequence, or fusing to another tail-fiber protein sequence, (via manipulation at the recombinant DNA level to produce the desired encoded protein) is done so that sequences in β turns on the same edge of the β-sheet are joined.

In addition to the above-described chimers, nanostructures of the invention can also comprise tail-fiber protein deletion constructs that are truncated at one end, e.g., are lacking an amino- or carboxy-end (of at least 5 or 10 amino acids) of the molecule. Such molecules truncated at the amino-terminus, e.g., of truncated gp37, gp34, or gp36, can be used to “cap” a nanostructure, since, once incorporated, they will terminate polymerization. Such molecules preferably comprise a fragment of a tail-fiber protein lacking at least the first 10, 20, or 60 amino terminal amino acids.

Generally, to adjust the length of the rod component proteins as desired, portions of the same or different tail-fiber proteins can be inserted into a tail-fiber chimer to lengthen the rod, or be deleted from a chimer, to shorten the rod.

Although T4 tail fibers are convenient for use as the protein rod body portion in the nanosensors of the present invention, other proteins that are sufficiently densely folded that they achieve the characteristics of a rigid rod may also be employed. By way of non-limiting examples, other viral fibers such as T4 short tail fiber (3 nm×35 nm) described by Burda, et. al. 2000. Stability of bacteriophage T4 short tail fiber. Biol. Chem. 381:225-228, and viral adhesins and tail fiber proteins, such as those described in Weigele, et al. 2003. Homotrimeric, b-standed vial adhesins and tail proteins. J. Bacteriol. 185:4022-4030 may be employed. Such viral fibers offer some of the advantages of T4 tail fibers, including stability, relative rigidity, and the ability to derivative the rod by recombinant technology. In addition, some of these options provide immediate access to recombinant production, and the ability to derivative the termini. For example, P22 tail spike is made recombinantly and purified as described in Haase-Pettingell, et al. 2001. Role for cysteine residues in the in vivo folding and assembly of the phage P22 tailspike. Protein Science 10:397-410. T4 P12 is produced recombinantly and purified as described in Jayaraman, et al. 1997. Thermal unfolding of bacteriophage T4 short tail fibers. Biotechnology Progress 13:837-843. Easier terminal derivatization is achievable with tail fibers because if one produces viral fibers using viral infection, this implies that the fiber must under some circumstance be competent for assembly/infectivity. Since these fibers often assemble to a capsid or other component at one end and provide surface recognition/binding on the other, terminal labeling can be difficult to accomplish. Thus if expression of a tail fiber/tailspike recombinantly, where ends can be derivatized/labeled/accessed without consideration for loss of assembly or recognition, facilitates end labeling fibers while the potential to control overall length is also expanded.

Also, viral fibers might be more likely to fold stably with large epitope inserts within the sequence, since other types of folds such as coiled-coils discussed below may be less stable in the presence of non-canonical inserts Hicks, et al. 2002. Investigating the tolerance of coiled-coil peptides to nonheptad sequence inserts. J. Struct. Biol. 137:73-81.

Dimeric/trimeric coiled coils may also be used as the protein rod body portion in the sensor of the invention. Such coils have been shown to have dimensions on the theo order of d=5 A, 1=1.5 A per AA, Lp˜10-30 nm (Creighton, T. E. 1993. Proteins: Structure and Molecular properties. pp. 182-198. W.H. Freeman and Company, New York.) An abundance of data is available on the design, expression and folding of coiled coil sequences (see Woolfson, D. N. 2005. The design of coiled-coil structures and assemblies. Advances in Protein Chemistry 70:79-112). Thus, designed/synthetic coiled coil protein rod body portions can be easily produced in a variety of consistent lengths and derivatized at the termini. This would allow for extensive tuning of hydrodynamic response since the rod length can be shortened for large recognition motif/analyte complexes, etc.

Single recombinant Fab having rod-like characteristics and dimensions of around a=4 nm×5 nm×8 nm have been described. Davies, et al. 1975. 3-Dimensional structure of immunoglobulins. Ann. Rev. Biochem. 44:639-667. Accordingly, antibodies/Ab fragments can also be employed as the protein rod body portion. In some embodiments, all antibody can serve the dual function of body portion and analyte binding portion. In others, the antibody can provide the ability to provide diverse and readily adjustable specificity by serving as a means for affixing an analyte binding moiety. For example, if the protein rod body portion is an antibody with specificity to rabbit antibodies, then rabbit-anti-analyte could be readily affixed to the sensor, and the specificity of the sensor would depend on the target of the rabbit anti-analyte.

### 7. Assembly of Individual Rod Components into Nanostructures

Expression of the proteins of the present invention in E. coli as described above results in the synthesis of quantities of protein, and allows the simultaneous expression and assembly of different components in the same cells. The methods for scale-up of recombinant protein production are straightforward and widely known in the art, and many standard protocols may be used to recover native and modified tail-fiber proteins from a bacterial culture.

In a preferred embodiment, native (non-recombinant) gp35 is isolated for use by growing up a bacteriophage T4 having an amber mutation in gene 36, in a su° bacterial strain (not an amber suppressor), and isolating gp35 from the resulting culture by standard methods. P34, P36-P37, P37, and chimers derived from them are purified from E. coli cultures as mature trimers. Gp35 and variants thereof are purified as monomers. Purification may be achieved by the following procedures or combinations thereof, using standard methods: 1) chromatography on molecular sieve, ion-exchange, and/or hydrophobic matrices; 2) preparative ultracentrifugation; and/or 3) affinity chromatography, using as the immobilized ligand specific antibodies or other specific binding moieties. For example, the C-terminal domain of P37 binds to the lipopolysaccharide of E. coli B. Other T4-like phages have P37 analogues that bind other cell surface components such as OmpF or TSX protein. Alternatively, if the proteins have been engineered to include heterologous domains that act as ligands or binding sites, the cognate partner is immobilized on a solid matrix and used in affinity purification. For example, such a heterologous domain can be biotin, which binds to a streptavidin-coated solid phase.

Alternatively, several components are co-expressed in the same bacterial cells, and subassemblies of larger nanostructures are purified subsequent to limited in vivo assembly, using one or more of the methods enumerated above.

The purified components may then be combined in vitro under conditions where assembly of the desired nanostructure occurs at temperatures between about 4° C. and about 37° C., and at pHs between about 5 and about 9. For a given nanostructure, optimal conditions for assembly (i.e., type and concentration of salts and metal ions) may be determined by routine experimentation, such as by changing each variable individually and monitoring formation of the appropriate products. Alternatively, one or more crude bacterial extracts may be prepared, mixed, and assembly reactions allowed to proceed prior to purification.

In some cases, one or more purified components assemble spontaneously into the desired structure, without the necessity for initiators. In other cases, an initiator is required to nucleate the polymerization of rods or sheets. This offers the advantage of localizing the assembly process (i.e., if the initiator is immobilized or otherwise localized) and of regulating the dimensions of the final structure. For example, rod components that contain a functional P36 C-terminus require a functional P37 N-terminus to initiate rod formation stoichiometrically; thus, altering the relative amount of initiator and rod component will influence the average length of polydisperse rod polymer.

In certain embodiments, the final nanostructure is composed of two or more components that cannot self-assemble individually, but only in combination with each other. In this situation, alternating cycles of assembly can be staged to produce final products of precisely defined structure (see Example 6B below).

When an immobilized initiator is used, it may be desirable to remove the polymerized unit from the matrix after staged assembly. For this purpose, specialized initiators may be engineered so that the interaction with the first rod component is rendered reversibly thermolabile (see Example 5). In this way, the polymer can be easily separated from the matrix-bound initiator, thereby permitting: 1) easy preparation of stock solutions of uniform parts or subassemblies, and 2) re-use of the matrix-bound initiator for additional cycles of polymer initiation, growth, and release.

In an embodiment in which a nanostructure is assembled that is attached to a solid matrix via gp34 (or P34), the nanostructure may be made detachable using a mutant (thermolabile) gp34 that can be made to detach upon exposure to a higher temperature (e.g., 40° C.). Such a mutant gp34, termed T4 tsB45, having a mutation near its C-terminal end such that P34 attaches to the distal tail-fiber half at 30° C., but can be separated from it in vitro by incubation at 40° C. in the presence of 1% SDS (unlike wild-type T4 which are stable under these conditions), has been reported (Seed, 1980, Studies of the Bacteriophage T4 Proximal Half Tail-fiber, Ph.D. Thesis, California Institute of Technology).

Proteins which catalyze the formation of correct (lowest energy) stable secondary (2°) structure of proteins are called chaperone proteins. Often, especially in globular proteins, this stabilization is aided by tertiary structure, e.g., stabilization of ″-sheets by their interaction in β-barrels or by interaction with alpha-helices. Normally chaperonins prevent intrachain or interchain interactions which would produce untoward metastable folding intermediates and prevent or delay proper folding. There are two known accessory proteins, gp57 and gp38, in the morphogenesis of T4 phage tail-fibers which are sometimes called chaperonins because they are essential for proper maturation of the protein oligomers but are not present in the final structures.

The usual chaperonin systems (e.g., groEL/ES) interact with certain oligopeptide moieties of the gene product to prevent unwanted interactions with oligopeptide moieties elsewhere on the same polypeptide or another peptide. These would form metastable folding intermediates which retard or prevent proper folding of the polypeptide to its native (lower energy) state.

Gp57, probably in conjunction with a membrane protein(s), may have the role of juxtaposing, and/or initiating the folding of 2 or 3 identical gp37 molecules. The gp38 protein may stabilize this interaction until the aligned peptides then zip up (while mutually stabilizing their nascent b-structures) to form a rod without further interaction with gp57 (Qu, et al., J. Bact., 2004, 186, S363-8369). Gp57 acts in T4 assembly not only for oligomerization of gp37 but also for gp34 and gp12.

### 8. Structural Components of Self Assembly of Struts In Vitro

As an alternative to starting the polymerization of chimers with the use of a preformed chimeric or natural oligomeric unit called an initiator produced in vivo, molecules (preferably peptides) that can self-assemble can be produced as fusion proteins, fused to the N- or C-terminus of tail-fiber variants of the invention (chimers, deletion/insertion constructs) to align their ends and thus to facilitate their subsequent unaided folding into oligomeric, stable 31-helical rod-like (rod) units in vitro, in the absence of the normally required chaperonin proteins (e.g., gp57) and host cell membrane proteins.

As an illustration, consider the P37 unit as an initiator of gp(37˜36) oligomerization and polymerization. Normally, proper folding of gp37 to a P37 initiator requires a phage infected cell membrane, and two chaperone proteins, gp38 and gp57. In a preferred embodiment, the need for gp38 can be obviated by use of a mutation, ts3813 (a duplication of 7 residues just downstream of the transition zone of gp37) which suppresses gene 38 (Wood, W. B., F. A. Eiserling, and R A. Crowther, 1994, “Long Tail-fibers: Genes, Proteins, Structure, and Assembly,” in Molecular Biology of Bacteriophage T4, (Jim D. Karam, Editor) American Society for Microbiology, Washington, D.C., pp 282-290). If a moiety that self-assembles into a dimer or trimer or other oligomer (“self-assembling moiety”) is fused to a C-terminal deletion of gp37 downstream or upstream of the transition region [the transition region is a conserved 17 amino acid residue region in T4-like tail-fiber proteins where the structure of the protein narrows to a thin fiber; see Henning et al., 1994, “Receptor recognition by T-even-type coliphages,” in Molecular Biology of Bacteriophage T4, Karam (ed.), American Society for Microbiology, Washington, D.C., pp. 291-298, and Wood et al., 1994, “Long tail-fibers: Genes, proteins, structure, and assembly,” in Molecular Biology of Bacteriophage T4, Karam (ed.), American Society for Microbiology, Washington, D.C., pp. 282-290], when it is expressed, the self-assembling moiety will oligomerize in parallel and thus align the fused gp37 peptides, permitting them to fold in vitro, in the absence of other chaperonin proteins.

Since P37 is a trimer, the self-assembling moiety can be a self trimerizing mutant leucine zipper peptide, pII in which both the a and d positions are substituted with isoleucine (Harbury P. B., et al. ibid.). Alternatively, a collagen peptide can be used as the self-assembling moiety, such as that described by Bella et al. (Bella, J.; Eaton, M.; Brodsky, B.; Berman, H. M. Science 1994, 226, 75-81), which self aligns by an inserted specific non-repeating alanine residue near the center.

### 9. Analyte Interacting Moieties

The sensors of the invention comprise an analyte interacting moiety or “display” which interacts with the analyte to result in a detectable change in hydrodynamic properties. In some embodiments of the invention, this analyte interacting moiety is suitably a peptide recognition sequence that provides for a specific binding interaction with a target analyte. The analyte interacting moiety may also include more transient and less specific interactions such as the interaction of coiled coils or viral adhesins with polysaccharides (Weigele et al. supra) or non-sequence specific interactions with nucleic acids (protein-DNA, protein-RNA as discussed generally in Choo et al. 2006 Binding proteins for the recognition of DNA. U.S. Pat. RE39,229. Published Feb. 29, 1996, intercalating agent-DNA and the like).

In other embodiments of the invention, the analyte interacting moiety is an enzyme that interacts with the analyte in a manner that changes the effective hydrodynamic properties of the sensor. In one such embodiment, the enzyme of the sensor interacts with analyte in the sample milieu to change the viscosity. For example, a sensor may comprise a hydrolase that cleaves specific sugar sequences/polysaccharides in the sample, reducing the sample viscosity and increasing the peak frequency of detection. The kinetics of hydrolysis might also be monitored in this fashion.

The interaction of the analyte with the sensor may also result in a cleavage of the sensor nowhere the analyte interacting moiety is a substrate for an analyte enzyme or other action on the sensor by the sample. The result of such cleavage will be two pieces, at least one of which is tagged with a magnetic particle. The tagged fragment of the cleaved sensor has a smaller hydrodynamic radius, and thus the peak in the complex magnetic susceptibility vs. frequency will shift to higher frequencies rather than lower frequencies. This is in a region of higher sensitivity for the measurement, because sensitivity should be essentially linear in frequency.

If magnetic nanoparticles are attached to both ends of the protein rod body portion that is cleaved, then two peaks should result, providing a more easily identifiable fingerprint of the cleavage. Two peaks will result as long as the hydrodynamic radii of the two fragments are unequal. This technique also provides for multiplexing. For example, if a protein rod body portion of a sensor is tagged magnetically at both ends and provided with multiple recognition moieties along its length, each one engineered for a different target, then the position of the cleavage, and hence the effective hydrodynamic radii of the fragments, would depend on the type of target attached. Thus one sensor body could be used for sensing the presence of multiple target molecules, viruses, proteins or large molecules.

The interaction of the analyte with the analyte interacting moiety may also be manipulated in situ to offer additional measurement/observation alternatives. For example, where the interaction is dependent of the presence of an ion, such as Ca⁺⁺, addition of a chelating agent such as EDTA or other agent that will sequester the ion will allow observation of the loss of the interaction and the return to a pre-analyte interaction hydrodynamic character.

### 10. “Displays” Engineered on the Strut

Previously we engineered a specific 23 amino acid ras epitope display in a manner that made it available to the surrounding medium. See Hyman, P. et al. Proc. Natl. Acad. Sci. USA 2000, 99, 4888-4893. This enabled its target, a monoclonal ras antibody, to bind tightly to the strut in solution. The generalization is that target specific binding peptides can serve to capture targets (e.g., molecules, viruses or cells) in solution. The following sections describe how one would design a protein strut with a non-native display (i.e., one that includes a non-native target recognition peptide sequence).

a. Identification and Characterization of a Large Deletion in P37

It is known that the tail-fiber acts as a trigger to signal initiation of the tail sheath contraction process that precedes phage DNA injection. The reversible, noncovalent binding of a number of tail-fiber distal ends to their specific receptor sites on the cell surface leads to a cooperative mechanical stress in the base plate. This stress triggers base plate expansion amid initiates the tail sheath contraction, which extends the tail core through the cell wall (Crawford, J. T.; Goldberg, E. G. J. Mol. Biol. 1977, 111, 305-313; and Crowther, R. A. J. Mol. Biol. 1980, 7, 159-174). The tail-fibers' critical function for phage viability provides a sensitive assay for rigidity in tail-fiber structure because any substantial loss of rigidity in the structure should impair the tail-fibers' triggering function.

We used PCR analysis to screen spontaneous pseudo-revertants of a gene 37 amber mutation (amA481), and identified a phage that appeared to have approximately 1 kb of DNA deleted from the middle of gene 37. This gene codes for the protein forming the distal end of the tail-fibers, and its C-terminus forms the phage receptor. Sequence analysis confirmed that a single contiguous segment of DNA coding for 346 of 1,026 amino acid residues (34%) was deleted in this phage, which was designated SΔ1 (spontaneous deletion 1). Table 1 shows the protein sequences of the deletion junctions and the corresponding wild-type protein. The deleted region begins at amino acid 73, which is 23 residues downstream from the conserved N-terminal domain of P37. This conserved region is thought to form the stiff butt end joint with the P36 C-terminal conserved domain (Riede, I.; Drexler, K.; Eschbach, M.-L. Nucleic Acids Res. 1985, 13, 605-616). Thus, this deletion falls completely within the P37 rod-like region. Phage carrying the SΔ1 mutation produce plaques of normal size and appearance indicating that they are able to infect and grow nominally. We also measured the adsorption rate of the SΔ1 phage (a measure of the rate of irreversible binding to the cell surface) and found that it was the same as wild-type phage (9.2 vs. 9.5×10⁻¹⁰ ml/min; SΔ1/wild-type=0.97). Since it was possible that the deletion mutation is compensated for by a second (duplication/insertion) mutation so that the overall tail-fiber length was unchanged, we cloned a 2-kb segment of DNA from the SΔ1 phage that surrounds the deletion site and placed it in a nonexpressing plasmid. Restriction and sequence analysis confirmed that this clone contained the expected DNA segments surrounding the 1,038 bp SΔ1 deletion and no additional DNA sequences. Homologous recombination was used to transfer the SΔ1 deletion into T4 phage containing the A481 amber mutation (which is located in the segment corresponding to the SΔ1 deletion segment). The SΔ1 deletion transferred at high efficiency, indicating that there is no other suppressor mutation needed to produce a viable phage.

In addition, we examined phage carrying the SΔ1 mutation by electron microscopy. The shortened distal portion of the tail-fiber is clearly visible in an electron micrograph of SΔ1 phage. To compare wild-type tail-fiber to SΔ1 tail-fiber we calculated the ratio of the lengths of the distal half fiber/proximal half fiber (D/P) by using measurements from enlarged electron micrographs. We found that for wild-type fibers D/P=0.99±0.06 (n=11) and for SΔ1 fibers D/P=0.54γ0.14 (n=6). This finding confirms that the viable SΔ1 phage have shortened but otherwise functional tail-fibers.

It will be appreciated this description is provided by way of example only and that other deletion sites, and other trod proteins can be employed. For example, a deletion can be formed in T4 P37 spanning amino acids A175-N544. The important factor is that the interserted peptide display be presented so that it can interact with a magnetic particle or a target analyte to form the sensor or sensor/analyte complex, as is the case when the PDL Is inserted in a β-loop of P37.

b. Inserting a Peptide Into a β-loop in P37

In the β-sheets forming the central rod regions of the tail-fibers, the loop regions contribute little to maintaining the H-bond network, nor to the van der Waals interaction in the hydrophobic layer within the rod (Branden, C. & Tooze, J. (1999) Introduction to Protein Structure (Garland, New York), 2nd Ed.; and Xu, G.; Wang, W.; Groves, J. T.; Hecht, M. II. Proc. Natl. Acad. Sci. USA 2001, 98, 3652-3657). We have shown that the loops can be more variable and flexible than other regions of the tail-fiber proteins, demonstrating that the junction of the SΔ1 deletion is in a loop (rather than in a β-strand) of the rod portion of gene 37. Surface loops in proteins can often be expanded to include additional peptide sequences with minimal effects on protein structure, function or stability (Regan, L. Curr. Opin. Strict. Biol. 1999, 9, 494-499). Thus, since the SΔ1 junction is in a loop, we can insert additional sequences into the junction, expanding the loop, without disrupting the structural integrity of the tail-fiber.

Towards this end we added DNA sequences encoding a penta-glycine peptide into the SΔ1 junction (Table 1, below) in the cloned gene segment. This modified sequence also transferred readily into phage by homologous recombination. [The SΔ1G5 phage produced poorer stocks, although the adsorption constant was almost the same as for the wild-type and SΔ1 phage (12×10⁻¹⁰ ml/min; SΔ1G5/wild type=1.3). Poorer stocks might indicate a mild interference with phage development.] This finding confirms that the SΔ1 junction is able to accept peptide insertions without any significant loss of structural integrity and supports our hypothesis that the junction identifies a loop in the restructure.

TABLE 1

Partial protein sequences of naturally
occurring and engineered gene 37 proteins

| | Partial protein sequence of gene | |
|---|---|---|
| Phage | 37 at SΔ1 junction | Seq ID No. |
| Wild-type T4 | GLLRLNGDYVQ//GSNNVQFYIDG | 1//2 |
| 37 SΔ1 | GLLRLNGD\|NVQFYADG | 3 |
| 37 SΔ1G5 | GLLRLNGDGGGGGNVQFYADG | 4 |
| 37 SΔ1ras1 (control) | GLLRLNGDGGGGARGVGKSALTIQLIGGGGNVQFYADG | 5 |
| 37 SΔras2 (mAb epitope) | GLLRLNGDGGGGEEYSAMRDQYMRTGEGGGGNVQFYADG | 6 |

Sequences flanking the SΔ1 junction are in italics, double slash represents 340 deleted amino acid residues, vertical line marks the position of the junction, inserted sequences are in boldface.

c. Inserting and Characterizing an Antibody Epitope into a Tail-Fiber Protein

To use tail-fiber derived proteins as mesoscale assembly units, one needs to attach specific functions to the assembled arrays of structural units. They may be attached before or after maturation of the final structure or at an intermediate step. The attachment may be covalent (e.g., disulfide bridges) or noncovalent (e.g., His tags). Incorporation of a peptide epitope may also be used to attach a functionality linked to the appropriate antibody. Fusions between antibodies and functional peptides have been extensively developed (Vitetta, E. S.; Fulton, R. I.; May, R. D.; Till, M.; Uhr, J. W. Science 1987, 238, 1098-1104; and Byers, V. S.; Baldwin, R. W. Immunology 1988, 65, 329-335). In the case of our nanoarchitectures, the compound would be fused to a mAb that is specific for an epitope in the structural unit. We have established that antibody epitopes can be incorporated into a tail-fiber protein.

For example, we have shown the insertion of two different 15 aa sequences from the human H-ras gene into the putative loop at the SΔ1 fusion junction (Table 1). Both peptides were flanked by four glycines on each side. One construct, SΔ1ras 1, containing a non-epitope segment of H-ras, was created as a control, whereas the other, SΔ1 ras2, contains the epitope specifically recognized by the rat monoclonal IgG antibody Y13-259 (Sigal, I. S., Gibbs, J. B.; D'Alonzo, J. S.; Scolnick, E. M. Proc. Natl. Acad. Sci. USA 1986, 83, 4725-4729). Each of these modified genes readily transferred into phage by homologous recombination.

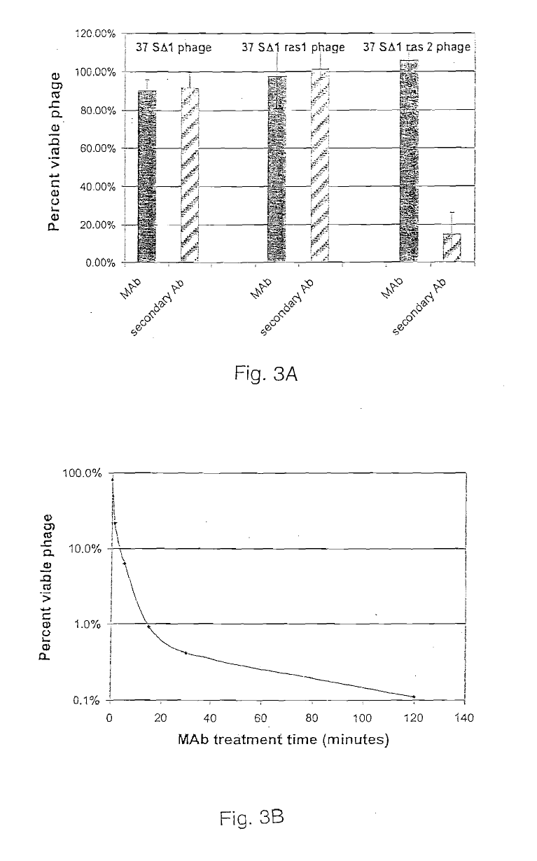

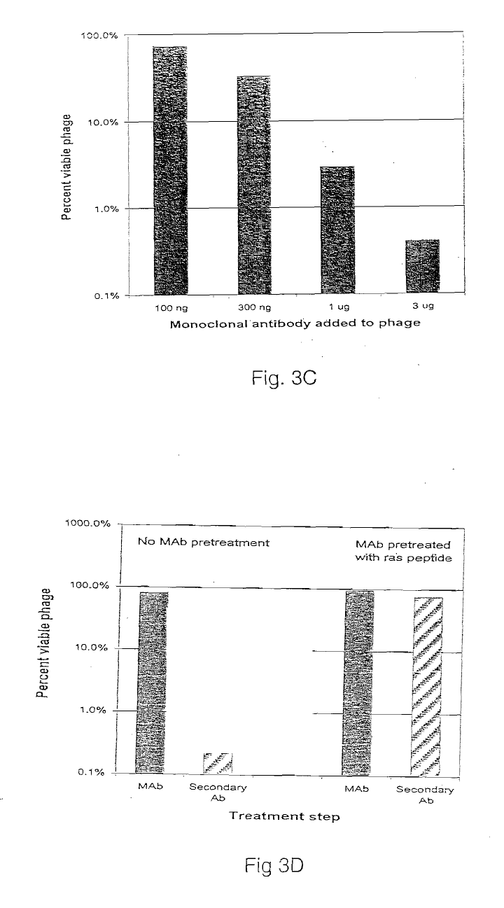

To demonstrate that the epitope was accessible for interactions with the exogenous antibody, we treated SΔ1, SΔ1ras1, and SΔ1ras2 phage with the anti-ras mAb. If the mAb can bind to the H-ras epitope, it might inactivate the phage by linking together tail-fibers on a single phage, thereby preventing proper binding to the cell surface. Alternatively, several phage might be linked together to form large non-infectious complexes. However, as FIG. 3A shows, mAb treatment alone (gray bars) did not result in phage inactivation. When the phage/mAb mixtures were further treated with an anti-rat IgG serum (striped bars) (which binds to the Fe region of the mAb), 85% of the SΔ1 ras2 phage were inactivated. Because the SΔ1ras1 control phage were unaffected and because the anti-rat IgG antiserum alone has no effect on the SΔ1 ras2 phage, this finding demonstrates that the ras2 epitope is exposed on the surface of the tailfiber and accessible to the mAb. The requirement for the secondary antibody for phage inactivation may reflect the axial symmetry of P37. Because each mature fiber contains more than one epitope in close proximity on each tail-fiber, it is likely that both binding sites in the mAb become bound to a single fiber. This would not be expected to inactivate the phage. Hence, the need for the secondary antibodies to crosslink tail-fibers by binding to two mAbs bound to two different fibers and inactivate the phage. Regardless of the specific mechanism of inactivation, these experiments show that a functional peptide can be added to the rod region of a tail-fiber protein without disrupting the tail-fiber structure or function.

We further investigated the interaction of the SΔ1ras2 phage with the mAb. FIG. 3B shows that inactivation depends on the time allowed for mAb binding before addition of the secondary antiserum, reaching a maximum of 99.9% by 120 min. FIG. 3C shows that inactivation also has a simple dose-response relationship with the amount of Y13-259 mAb used. FIG. 3D shows that the SΔ1ras2 phage could be protected from inactivation by pretreating the mAb with a free 15-aa peptide of the same sequence as the 15-residue epitope inserted into the tail-fiber protein. Although 99.8% of the phage were inactivated in the control treatment (with buffer only), there was no significant inactivation when the mAb was pretreated with the peptide. This finding demonstrates that the inactivation requires a specific interaction of the antibody with its specific epitope sequence.

We examined how the mAb interacts with the tail-fiber by imaging mAb-treated phage. The phage form a “bouquet” with the tail-fibers linked together. It is unlikely that phage in such a bouquet could orient properly on the cell surface to allow the tail-fibers to function cooperatively and trigger infection. Taken together, these results demonstrate that rearrangements, fusions, and insertions can be made to a tail-fiber protein without disrupting the functional integrity of the mature protein structure. They also support our hypothesis that fusion sites can be used for insertion of foreign peptides in such a way that they are available for binding. Further, these results support our hypothesis that the binding domain at the N-terminal end of P37 (and, presumably, the binding domains of other tail-fiber proteins) is functionally separable from the central rod region. This finding suggests that chimeric proteins composed of the P37-binding domain of P36 joined by a central rod domain to the P36-binding end of P37 will form homo-polymeric fibers. The fusion site of these chimeric proteins should accept a functional peptide just as the SΔ1 junction does.

This will provide the potential for attaching immunoconjugated functional moieties at precise locations along the fiber. The length of the chimeric proteins can be adjusted by using more or less of the rod region from either of the parent proteins, allowing the spacing of the functional moieties to be controlled. Further, other β-loops within the central rod domain can be used as insertion sites for the addition of antigenic peptides that can subsequently be recognized by antibodies to add either functional or structural capabilities including crosslinking of the polymeric fibers into open two- and three-dimensional arrays.

This approach allows one to engineer protein fibers to place functional moieties in predesigned positions relative to one another to construct nanocomponents and nanodevices that exhibit functions not attainable with single nanoparticles or nanostructured materials. Taken together, the capability demonstrated here provides great potential for fabrication of a broad range of nanostructures.

### 11a. Paramagnetic Nano-Particle Components to be Bound by Display

Magnetic spherical particles with a diameter of less than about 10 nm are magnetic mono domains both in a magnetic field and in the zero field. A particle being a magnetic mono domain means that the particle only contains one magnetization direction. Depending on the size, geometry, temperature, measurement time, magnetic field and material of the particles, they can either be thermal blocked or super paramagnetic. The direction of the magnetization for thermal blocked particles is oriented in a manner in the magnetic particle relative to the crystallographic orientation of the particle, and “locked” to this direction. Under the influence of an external magnetic field, the entire particle physically rotates so that its magnetization directions gradually coincide to some extent with the direction of the external added field.

Small magnetic particles can be manufactured from a variety of materials, for example Fe₃O₄, Fe₂O₃, cobalt-doped iron oxide or cobalt iron oxide (CoFe₂O₄). Other magnetic materials, specifically (but not exclusively) rare earth metals (for example ytterbium or neodymium), their alloys or compounds containing rare earth metals, or doped magnetic (element) substances are also possible. The sizes of the particles can be produced from about 3 nm to about 30 nm. The final size in this process depends on a number of different parameters during the manufacturing.

Magnetic nano-particles offer some attractive possibilities as components of nanosensors. First, they have controllable sizes ranging from a few nanometers up to tens of nanometers, which places them at dimensions that are smaller than or comparable to those of a cell (10-100 μm), a virus (20450 nm), a protein (5-50 nm) or a gene (2 nm wide and 10-100 nm long). This means that they can ‘get close’ to a biological entity of interest. Indeed, they can be coated with biological molecules to make them interact with or bind to a biological entity, thereby providing a controllable means of ‘tagging’ or addressing it. The nano-particles are magnetic and can be manipulated by an external magnetic field gradient. This ‘action at a distance’, combined with the intrinsic penetrability of magnetic fields into human tissue, opens up many applications involving the transport and/or immobilization of magnetic nano-particles, or of magnetically tagged biological entities. In this way, they can be made to deliver a package, such as an anticancer drug, or a cohort of radionuclide atoms, to a targeted region of the body, such as a tumor. Third, the magnetic nano-particles can be made to respond to a time-varying magnetic field, with advantageous results related to the transfer of energy from the exciting field to the nanoparticle. For example, if the particle is metallic, it can be made to heat up, which leads to its use as hyperthermia agents, delivering toxic amounts of thermal energy to targeted bodies such as tumors; or as chemotherapy and radiotherapy enhancement agents, where a moderate degree of tissue warming results in more effective malignant-cell destruction. These, and many other potential applications, are made available in biomedicine as a result of the special physical properties of magnetic nano-particles.

In the instant invention, in a preferred embodiment, the magnet (reporter) component of the nano-sensor is a magnetic nanoparticle. For example, a wide array of mono-disperse nanoparticles, with controlled size and magnetics, can be attached to the nano-sensors by specific high affinity PDLs to bind to the magnet and engineer the peptide to extend from termini of the high melting trimeric tetraheptad coiled-coil (Qu, Y. et al. J. Bacteriol. 2004, 186, 8363-8369).

We expect high avidity of magnet-rod binding because each of the three C-terminal peptides should bind to the same magnet. If the binding is not tight enough, and alternative is to coat the magnet with streptavidin and incorporate a 15 residue biotin analogue, Kd=4 nM (Lamla, T.; Erdmann, V. A. Protein Expression and Purification 2004, 33, 39-47).

In one embodiment the nano-magnets are magnetic nanoparticles comprised of one or more metals. In one embodiment, the nano-magnets are magnetic nanoparticles comprised of one or more transition metals. In a preferred embodiment, the nano-magnets are selected from magnetic nanoparticles consisting of one or more of the following metals or metalloids: cobalt, iron, gold, chromium, palladium, platinum, manganese, neodymium, nickel, zirconium, copper, niobium, boron and their oxides and alloys. In a more preferred embodiment the magnetic nanoparticles are iron-based metals and alloys, and their oxides and nitrides (e.g., iron, iron-gold, iron-chromium, iron nitride (Fe—N), iron oxide (Fe₃O₄), iron-palladium, iron-platinum, and iron-neodymium-boron, iron-neodymium-boron-niobium-copper, ironzirconium-neodymium-boron.) In certain embodiments, the nano-magnet is an transition-metal-containing biological entity. In a preferred embodiment, the nano-magnet is all iron-containing biological entity (e.g. ferritin).

In a preferred embodiment, the nano-magnets are monodisperse MFe₂O₄, wherein M is iron, cobalt or manganese. In one embodiment, the magnetic nanoparticle has a diameter between about 0.1 nm and 100 nm. In a preferred embodiment, the magnetic nanoparticle has a diameter between about 1 nm and about 50 nm. In a preferred embodiment, the magnetic nanoparticle has a diameter between about 7 nm and about 15 nm. In a preferred embodiment, the magnetic nanoparticle has a diameter of about 1 nm.

Nanoparticles may be coated with a material to aid in their interaction with the nanostrut. In one embodiment, the magnetic nanoparticle is coated with a polymer. In another embodiment, the nanoparticle is coated with a biological polymeric material. In yet another embodiment, the magnetic nanoparticle is coated with nucleic acids, or oligosaccharides, or proteins.

### 11b. “Target” Component to be Bound by Display

In one embodiment, the nano-sensors contain target recognition peptides specific to a biological molecule or chemical entity. A “biological molecule” is broadly defined as a molecule which has been constructed from the compounds from which organisms are formed. Such compounds can be amino acids, nucleic acids, saccharides, membrane lipids, or biological cofactors.

Amino acids include the twenty essential amino acids and other amino acids which can be incorporated into proteins. Molecules constructed from amino acids also include peptides, i.e., chains composed of amino acids linked together through peptide bonds. These can be molecules such as neurotransmitters, hormones, and/or peptides derived from the functional parts of larger peptides. Molecules constructed from amino acids also include proteins, i.e., longer chained peptides which may have one of many noncatalytic functions, such as electron transfer proteins (e.g., ferredoxins and flavodoxins), immune protection proteins (antibodies), proteins that generate or transmit nerve impulses (e.g., acetylcholine, dopamine, and the rhodopsin membrane receptors), structural proteins (e.g., collagen, fibrin, glycoproteins, elastin, etc.), other binding proteins (e.g., histones), and mass transport proteins (e.g., ferritin, hemoglobin).

Saccharides include monosaccharides (e.g., glucose, fructose), oligosaccharides (e.g., sucrose, and raffinose), and polysaacharides (e.g., starch, and cellulose). Membrane lipids include molecules such as phospholipids (e.g., lipid bilayer membranes and other fatty acids), glycolipids, cholesterol and its derivatives, and prostaglandin and its derivatives. Nucleic acids include the five common nucleotides (adenine, guanine, cytosine, uracil, and thymine), oligonucleotides, and polynucleotides or nucleic acids such as DNA, m-RNA, and t-RNA.

Cofactors are biological molecules whose catalytic function may not be generated or specifically directed until associated with a polypeptide chain. Examples are riboflavin derivatives, porphyrins, thiamin pyrosphosphate and nicotinamide adenine dinucleotide. Since specific interactions usually occur in biological systems it is probable that the sensor can have a distinguished role within this area, for example analysis of biochemical markers for different diseases. Examples of molecules that can inter-act specifically with each other are: a) antibody-antigen; b) receptor-hormone; c) two complementary single strands of DNA, and d) enzyme-substrate/enzyme-inhibitor.

### 12. Methods of Use

One aspect of the invention relates to detecting changes in the magnetic response of the magnetic particles that depend on the Brownian relaxation (τ) in a carrier fluid (for example, water or a suitable buffer fluid, or another fluid suitable for the biomolecules that are the final target for the detection) under influence of an external AC-magnetic field. At the modification of the effective volume of the particles or their interaction with the surrounding fluid (e.g., when biomolecules or antibodies are bound on their surfaces) the hydrodynamic volume of respective particles will be increased resulting in a decrease of the frequency, fₘₐₓ, wherein the out-of-phase component of the magnetic susceptibility is at its maximum. Hence, the initially mentioned method further involves modification of the effective volume of the particle or its interaction with the carrier fluid as the hydrodynamic volume of the particle changes. This implies a change of frequency where the out-of-phase component of the magnetic susceptibility has its maximum. The measurement is actually a relative measurement because changes in a modified particle system are compared with an original system. Therefore, at least two sample containers and two detector coils are used for the measurement. The amplitude and phase of the voltage output from the secondary coil set is a measure of the magnetic susceptibility of the specimen to be analyzed.

An external oscillator/frequency generator can be arranged, such that the coils are in a bridge circuit. The difference between the induced voltage output of both detector coils is measured, and, in addition, the phase difference between it and that of the frequency generator is measured. A noise source can be used as well and the response of the system can be analyzed by means of a FFT (Fast Fourier Transform) analysis of an output signal. According to one embodiment, the signal difference is set to zero between the secondary coils, which is done through adjustment of the passive resistance and inductive components of an external (nulling) circuit attached to one of the secondary coils. The zero setting can be done through minimizing, the signal through adding a determined amount of a magnetic sensors in one of the spaces wherein the sample containers are placed, so that the substance creates an extra contribution to the original signal that therefore can be set to zero.

FIGS. 2 A and B show a schematic representation of device capable of making this measurement. In FIG. 2A, the primary coil 201 and its associated circuitry are separated from the secondary coils 202 and 203, and the capillary 204 is not shown for clarity. FIG. 2B shows cross section through secondary coil 202 depicting the relative positions of the primary coil 201, the secondary coil 202 and the capillary 204 having sample space 205. In use, an ac current is passed through coil 201 of the primary circuit, resulting in the generation of an oscillating magnetic field within the coil. The strength of this field depends on the magnitude of the signal. At the correct frequency, the magnetic field results in an oscillation of sensor molecules within the sample chambers α and β of the capillary 204. Sample chamber α is disposed within secondary coil 202 and sample chamber β coils is disposed within secondary coil 203. Secondary coils 202 and 203 are in series, but are wound in series opposition (opposite directions).

Because of the oscillations of the sensors, a change in magnetic field occurs that induces an ac current in the secondary coils 202 and 203. When both sample chamber at and β contain the same thing, the frequency shift should be the same. Null electronic circuitry N is used is balance the signal from the secondary coils 202 and 203 prior to the introduction of sample into one of the chambers to account for instrumental % variation.

When a test sample is introduced into one of the chambers, α or β, if analyte is present and associated with the sensor, it will cause a lengthening of the relaxation time and hence a decrease in the frequency at which the out-of-phase (loss) component of the magnetic susceptibility occurs for that coil. This phase difference is detected using a conventional phase sensitive detector (φ).

In the alternative, sample potentially containing analyte can be added to both sample chambers. The two chambers are then titrated, one with a control solution and the other with the control solution containing a competitor for the binding of the sensor to the analyte. If analyte is present, this will result in sensor being freed on one side of the chamber as a result of competitive binding, but not on the other. The resulting change in the magnetic properties of the two chambers can therefore be observed in the same manner, although the change will be in the opposite direction.

The fluid sample comprises one or several proteins in a fluid solution, like blood, blood plasma, serum, saliva, feces, urine or samples from water, waste, or soil (for environmental testing). The analysis can be connected to the particle through interaction with a second molecule, which is connected to the particle before the analysis starts. Interactions that may be probed include antibody-antigen, receptor-hormone, two complementary single strings of DNA and enzyme-substrate/enzyme-inhibitor.

The invention also relates to a method for detection of changes in the magnetic response of at least one magnetic particle provided with an external layer in a carrier fluid, which method comprises measurements of the magnetic particles characteristic rotation period with respect to the agitation of the external layer. The method uses at least two substantially identical detection coils connected to detection electronics and sample containers for absorbing carrier fluid. An excitation coil can surround the detection coils and sample containers for generation of a homogeneous magnetic field at the sample container. According to one embodiment, the excitation coil, measurement coils and sample container are placed concentric and adjusted round its vertical center axis. The arrangement can furthermore comprise an oscillator system wherein the detection coils constitute the frequency-determining element in an oscillator circuit.

The coils are arranged in the oscillator return coil. The coils that surround the samples respectively are electrically phase-shifted versus each other (series opposing), so that when properly balanced by the nulling circuit, the output of the system measured by a phase sensitive detector, is zero (both in- and out-of-phase components). Additionally, an operation amplifier can be arranged to subtract two voltages from each other. Consequently when two samples with unequal magnetic response are inserted in the two coils, the output signal is non-zero. The arrangement comprises a phase-locking circuit in one embodiment.

In a second embodiment the arrangement comprises oscillator/frequency generator signal to generate period variable current to excite the coils by means of white noise. Frequency depending information is received through an FFT-filtering of the response.

### 13. Applications

Nanosensors according to the invention have applications in many different areas. An important area is that of clinical medicine, where numerous analytical instruments and techniques are currently employed to determine the concentration of clinically important markers. Until recently, radioimmunoassay techniques were used for most chemical analyses, but these require typically large and expensive instruments designed for large centralized hospital or clinical laboratories. Pressures to cut health-care costs are creating more demand for less expensive, smaller analyzers which can be used in decentralized organizations, e.g., in individual hospital wards, outpatient departments, and physicians' offices. The nanosensors of the invention, as a result of their small size, low cost, selectivity, and sensitivity, may fill that need. Various chemical sensors exist for potassium, sodium, hydrogen, lithium, and calcium ions, but have yet to be developed for most proteins, hormones, metabolites, and organic drugs.

In another embodiment, the sensor cans for example, be used within medical diagnostics. The biosensor could for example replace some ELISA analysis (Enzyme Linked Immunosorbent Assay). This method is used today to a great extent to determine contents of biochemical markers (for example proteins) found in complex body fluids, such as blood, serum and cerebro-spinal fluid. Examples of ELISA analysis that can replace the new biosensor are: a) analysis of tau proteins in cerebro-spinal fluid (part of diagnosis of Alzheimer's disease); b) analysis of PSA in serum (diagnosis of prostate cancer); c) analysis of acute phase proteins measured in connection with heart disease; and d) analysis of CA 125 in serum (diagnosis of cancer in the ovaries).

Veterinary health care is another area having needs similar to those of the human-healthcare field. Biosensors have many potential applications in the diagnosis and monitoring of animal health problems.

Another large area of use for the biosensors is that of fermentation control (Karube, I. Biotechnology and Genetic Engineering Reviews 1984, 2, 313). There are many industrial applications of biochemical and microbiological processes in fields such as the production of food, pharmaceuticals, wastewater treatment and energy production. Fermentation reactions also have an important role in such biotechnological processes. It is necessary to control carefully the systems involved to optimize production. Rapid and sensitive on-line monitoring and control of reactant and product concentrations, reaction conditions and the like call for sensors specific to the substrates and products of fermentation.

Environmental monitoring is another growing area wherein biosensors are needed (Neujahr, H. Y. Biotechnol. Genet. Eng. Rev. 1984, 1, 167-186). Rising concerns over atmospheric, water, and soil pollution are creating a demand for chemical sensors to monitor substances such as pesticides, phenols, phenoxyacids, nitrilotriacetate, heavy metals, nitrate, phosphate, sulphate, and urea.

The defense industry also has a need for sensitive chemical sensors to monitor trace levels of chemical and biological warfare agents. Other applications for chemical sensors include food and feed process and quality control, agricultural diagnostics and monitoring, industrial hygiene, and toxicology testing.

Importantly, the sensors and methods of the present invention can be used for “low throughput screening”, that is the accomplishment of one or several analyses al the same time, or for “high throughput screening”, that is the accomplishment of a large number of analyses simultaneously.

### EXAMPLES

The following examples are intended to illustrate the present invention without limiting its scope. In the examples below, all restriction enzymes, nucleases, ligases, etc. are commercially available from numerous commercial sources, such as New England Biolabs (NEB), Beverly, Mass.; Life Technologies (GIBCO-BRL), Gaithersburg, Md.; and Bochringer Mannheim Corp. (BMC), Indianapolis, Ind.

### Example 1

### Design, Construction and Expression of Internally Deleted P37

The gene encoding gp37 contains two sites for the restriction enzyme Bgl II, the first cleavage occurring after nucleotide 293 and the second after nucleotide 1486 (the nucleotides are numbered from the initiator methionine codon ATG.) Thus, digestion of a DNA fragment encoding gp37 with Bgl II, excision of the intervening fragment (nucleotides 294-1485) and religation of the 5′ and 3′ fragments results in the formation of an internally deleted gp37, designated ΔP37, in which arginine-98 is joined with serine-497.

The restriction digestion reaction mix contains: 2 μL of gp37 plasmid DNA (1 μg/μL), 1 μL NEB buffer #2 (10×), 6 μL H₂O (1 μL), and 1 μL Bgl II (10 U/μL). The gp37 plasmid signifies a pT7-5 plasmid into which gene 37 has been inserted in the multiple cloning site, downstream of a good ribosome binding site and of gene 57 to chaperone the trimerization. The reaction is incubated for 1 h at 37° C. Then, 89 μL of T4 DNA ligase buffer and 1 μL of T4 DNA ligase are added, and the reaction is continued at 16° C. for 4 hours.

2 μL of the Stu I restriction enzyme are then added, and incubation continued at 37° C. for 1 h. (The Stu I restriction enzyme digests residual plasmids that were not cut by Bgl II in the first step, reducing their transformability by about 100-fold.)

The reaction mixture is then transformed into E. coli strain BL21, obtained from Novagen, using standard procedures. The transformation mixture is plated onto nutrient cigar containing 100 μg/ml ampicillin, and the plates are incubated overnight at 37° C.

Colonies that appear after overnight incubation are picked, and plasmid DNA is extracted and digested with Bgl II as above. The restriction digests are resolved on 1% agarose gels. A successful deletion is evidenced by the appearance after gel electrophoresis of a new DNA fragment of 4.2 kbp, representing the undeleted part of gene 37 which is still attached to the plasmid and which re-formed a BglII site by ligation. The 1.2 kbp DNA fragment bounded by Bgl II sites in the original gene is no longer in the plasmid and so is missing from the gel.

Plasmids selected for the predicted deletion as above are transformed into E. coli strain BL21(DE3). Transformants are grown at 30° C. until the density (A600) of the culture reaches 0.6 IPTG is then added to a final concentration of 0.4 mM and incubation is continued at 30° C. for 2 h, after which the cultures are chilled on ice. 20 μL of the culture is then removed and added to 20 μL of a two-fold concentrated “cracking buffer” containing 1% sodium dodecyl sulfate, glycerol, and tracking dye. 15 μL of this solution are loaded onto a 10% polyacrylamide gel; a second aliquot of 15 μL is first incubated in a boiling water bath for 3 min and then loaded on the same gel. After electrophoresis, the gel is fixed and stained. Expression of the deleted gp37 is evidenced by the appearance of a protein species migrating at an apparent molecular mass of 65-70,000 daltons in the boiled sample. The extent of trimerization is suggested by the intensity of higher-molecular mass species in the un-boiled sample and/or by the disappearance of the 65-70,000 dalton protein band.

The ability of the deleted polypeptide to trimerize appropriately is directly evaluated by testing its ability to be recognized by an anti-137 antiserum that reacts only with mature P37 trimers, using a standard protein immunoblotting procedure.

An alternative assay for functional trimerization of the deleted P37 polypeptide (ΔP37) is its ability to complement in vivo a T4 37-phage, by first inducing expression of the ΔP37 and then infecting with the T4 mutant, and detecting progeny phage.

A ΔP37 was prepared as described above, and found capable of complementing a T4 37-phage in vivo.

### Example 2

### Design, Construction and Expression of a gp37-36 Chimer

The starting plasmid for this construction is one in which the gene encoding gp37 is cloned immediately upstream (i.e., 5′) of the gene encoding gp36. The plasmid is digested with Hae III, which deletes the entire 3′ region of gp37 DNA downstream of nucleotide 724 to the 3′-terminus, and also removes the 5′ end of gp36 DNA from the 5′ terminus to nucleotide 349. The reaction mixture is identical to that described in Example 1, except that a different plasmid DNA is used, and the enzyme is HaeIII. Ligation using T4 DNA ligase, bacterial transformation, and restriction analysis are also performed as in Example 1. In this case, excision of the central portion of the gene 37-36 insert and religation reveals a novel insert of 346 in-frame codons, which is cut only once by HaeIII (after nucleotide 725). The resulting construct is then expressed in E. coli BL21 (DE3) as described in Example 1.

Successful expression of the gp37-36 chimer is evidenced by the appearance of a protein product of about 35,000 daltons. This protein will have the first 242 N-terminal amino acids of gp37 fused to the final 104 C-terminal amino acids of gp36 (numbered 118-221) The utility of this chimer depends upon its ability to trimerize and attach end-to-end. That is, carboxy termini of said polypeptide will have the capability of interacting with the amino terminus of the P37 protein trimer of bacteriophage T4 and to form an attached diner, and the amino terminus of the trimer of said polypeptide will have the capability of interacting with other said chimer polypeptides. This property can be tested by assaying whether introduction of ΔP37 initiates trimerization and polymerization. Alternatively, polyclonal antibodies specific to P36 trimer may be used to detect P36 subsequent to initiation of trimerization by ΔP37.

A gp37-36 chimer was prepared similarly to the procedures described above, except that the restriction enzyme TaqI was used instead of HaeIII. Briefly, the 5′ fragment resulting from TaqI digestion of gene 37 was ligated to the 3′ fragment resulting from TaqI digestion of gene 36. This produced a construct encoding a gp37-36 chimer in which amino acids 1-48 of gp37 were fused to amino acids 100-221 of gp36. This construct was expressed in E. coli BL21 (DE3), and the chimer was detected as an 18 kD protein. This gp37-36 chimer was found to inhibit the growth of wild type T4 when expression of the gp37-36 chimer was induced prior to infection (in an in vitro phage inhibition assay).

### Example 3

### Mutation of the GP37-36 Chimer to Produce Complementary Suppressors

The goal of this construction is to produce two variants of a trimerizable P37-36 chimer: One in which the N-terminus of the polypeptide is mutated (A, designated *P37-36) and one in which the C-terminus of the polypeptide is mutated (B, designated P37-36*). The requirement is that the mutated *P37 N-terminus cannot form a joint with the wild-type P36 C-terminus, but only with the mutated *P36 N-terminus. The rationale is that A and B each cannot polymerize independently (as the parent P37-36 protein can), but can only associate with each other sequentially (i.e., P37-36*+*P37-36% P37-36*&*P37-36).

A second construct, *p37-P36*, is formed by recombining *P37-36 and P37-36* in vitro. When the monomers *gp37-36* and gp37-36 are mixed in the presence of P37 initiator, gp37-36 would trimerize and polymerize to (P37-36)n; similarly, *P37 would only catalyze the polymerization of *gp37-36* to (*P37-36*)n. In this case, the two chimers could be of different size and different primary sequence with different potential side-group interactions, and could initiate attachment at different surfaces depending on the attachment specificity of P37.

The starting bacterial strain is a su° strain of E. coli (which lacks the ability to Suppress amber mutations). When this strain is infected with a mutant T4 bacteriophage containing amino mutations in genes 35, 36, and 37, phage replication is incomplete, since the tail-fiber proteins cannot be synthesized. When this strain is first transformed with a plasmid that directs the expression of the wild type gp35, gp36 and gp37 genes and induced with IPTG, and subsequently infected with mutant phage, infectious phage particles are produced; this is evidenced by the appearance of “nibbled” colonies. Nibbled colonies do not appear round, with smooth edges, but rather have sectors missing. This is caused by attack of a microcolony by a single phage, which replicates and prevents the growth of the bacteria in the missing sector.

For the purposes of this construction, the 3′-terminal region of gene 36 (corresponding to the C-terminal region of gp36) is mutagenized with randomly doped oligonucleotides. Randomly doped oligonucleotides are prepared during chemical synthesis of oligonucleotides, by adding a trace amount (up to a few percent) of the other three nucleotides at a given position, so that the resulting oligonucleotide mix has a small percentage of incorrect nucleotides at that position. Incorporation of such oligonucleotides into the plasmid will result in random mutations (Hutchison, C. A. III et al. Methods. Enzymol. 1991, 202, 356-390).

The mutagenized population of plasmids (containing, however, unmodified genies 36 and 37), is then transformed into the su° bacteria, followed by infection with the mutant T4 phage as above. In this case, the appearance of non-“nibbled” colonies indicates that the mutated gp36 C-termini can no longer interact with wild type P37 to form functional tail-fibers. The putative gp36* phenotypes found in such non-nibbled colonies are checked for lack of trimeric N-termini by appropriate immunospecificity as outlined above, and positive colonies are used as source of plasmid for the next step.

Several of these mutated plasmids are recovered and subjected to a second round of mutagenesis, this time using doped oligonucleotides that introduce random mutations into the N-terminal region of gp37 present on the same plasmid. Again, the (now doubly) mutagenized plasmids are transformed into the supo strain of E. coli and transformants are infected with the mutant T4 phage. At this stage, bacterial plates are screened for the re-appearance of “nibbled” colonies. A nibbled colony at this stage indicates that the phage has replicated by virtue of suppression of the non-functional gp36* mutation(s) by the *P37 mutation. In other words, such colonies must contain novel *P37 polypeptides that have now acquired the ability to interact with the P36* proteins encoded on the same plasmid.

The *P37-36 and P37-36* paired suppressor chimers (A and B as above) are then constructed in the same manner as described in Example 2. In this case, however, *P37 is used in place of wild type P37 and P36* is used in place of wild type P36. A *P37-36* chimer can now be made by restriction of *P37-36 and P37-36* and religation in the recombined order. The *P37-36* can be mixed with the P37-36 chimer, and the polymerization of each can be accomplished independently in the presence of the other. This is useful when the rod-like central portion of these chimers have been modified in different ways.

### Example 4

### Design, Construction and Expression of a gp36-34 Chimer

The starting plasmid for this construction is one in which the vector containing gene 57 and the gene encoding gp36 is cloned immediately upstream (i.e., 5′) of the gene encoding gp34. The plasmid is digested with NdeI, which cuts after bp 219 of gene 36 and after bp 2594 of gene 34, thereby deleting the final 148 C-terminal codons from the gp36 moiety and the first 865 N-terminal codons from the gp34 moiety. The reaction mixture is identical to that described in Example 1, except that a different plasmid DNA is used, and the enzyme used is NdeI (NEB).

Ligation using T4 DNA ligase, bacterial transformation, and restriction analysis are also performed as in Example 1. This results in a new hybrid gene encoding a protein of 497 amino acids (73 N-terminal amino acids of gp36 and 424 C-terminal amino acids of gp34, numbered 866-1289.)

As an alternative, the starting plasmid is cut with SphI at bp 648 in gene 34, and the Exo-Size Deletion Kit (NEB) is used to create deletions as described above. The resulting construct is then expressed in E. coli BL21(DE3) as described in Example 1. Successful expression of the gp36-34 chimer is evidenced by the appearance of a protein product of about 55,000 daltons. Preferably, the amino termini of the polypeptide homotrimer have the capability of interacting with the gp35 protein, and then the carboxy termini have the capability of interacting with other attached gp35 molecules. Successful formation of the trimer can be detected by reaction with anti-P36 antibodies or by attachment of gp35 or by the in vitro phage inhibition assay described in Example 2.

### Example 5

### Assembly of One-Dimensional Rods

### A. Simple Assembly

The P37-36 chimer described in Example 2 is capable of self-assembly, but requires a P37 initiator to bind the first unit of the rod. Therefore, a P37 or a ΔP37 trimer is either attached to a solid matrix or is free in solution to serve as an initiator. If the initiator is, attached to a solid matrix, a thermolabile P37 trimer is preferably used. Addition of an extract containing gp37-36, or the purified gp37-36 chimer, results in the assembly of linear multimers of increasing length. In the matrix-bound case, the final rods are released by a brief incubation at high temperature (40-60° C., depending on the characteristics of the particular thermolabile P37 variant.) The ratio of initiator to gp37-36 can be varied, and the size distribution of the rods is measured by any of the following methods: 1) Size exclusion chromatography; 2) Increase in the viscosity of the solution; and 3) Direct measurement by electron microscopy.

### B. Staged Assembly

The P37-36 variants *P37-36 and P37-36* described in Example 3 cannot self-polymerize. This allows the staged assembly of rods of defined length, according to the following protocol:

1. Attach initiator P37 (preferably thermolabile) to a matrix.

2. Add excess *gp37-36 to attach and oligomerize as P37-36 homooligomers to the N-terminus of P37.

3. Wash out unreacted *gp37-36 and flood with gp37-36*.

4. Wash out unreacted gp37-36* and flood with excess *gp37-36.

5. Repeat steps 2-4, n−1 times.

6. Release assembly from matrix by brief incubation at high temperature as above.

The linear dimensions of the protein rods in the batch will depend upon the lengths of the unit heterochimers and the number of cycles (n) of addition. This method has the advantage of insuring absolute reproducibility of rod length and a homogenous, monodisperse size distribution from one preparation to another.

### Example 6

### Design of Protein Struts for Self-Assembling Nanoconstructs

### A. E. coli and Phage Strains and Reversion Assay

T4 37amA481 (Fisher, K. M.; Bernstein, H. Mol. Gen. Genet. 1970, 106, 139-150) vas the mutant used to derive all phage strains discussed herein. E. coli B40 (suI) (lab strain, Courtesy of P. Strigini, Harvard Medical School, Boston) was used to grow and titer phage containing an amber mutation, and E. coli BB (su0) (McFall, E.; Stent, G. W. J. Gen. Microbiol. 1970, 18, 346-363) was used for all non-amber phages. T4 37amA481 pseudorevertants were identified by their ability to form plaques on BB, and stocks were prepared by standard techniques (Carlson, K. & Miller, E. S. (1994) in Molecular Biology of Bacteriophage T4, ed. Karam, J. D. (Am. Soc. Microbiol. Press, Washington, D.C.), pp. 421-441). Plasmids were produced, and recombined with phage using E. coli MC1061 (F-araD139 !(ara-leu)7696 galE15 galK16 !(lac)X74 rpsL (Strr) hsdR2 (rK− mK+) mcrA mcrB1) (14) as the host strain.

### B. PCR Primers and Product Cloning

Primers cysF (CTATTAACGGACTTTTGAGA, Seq. ID No. 7) and cysR (TTCAATACGTCCAATAGTTT, Seq. ID No. 8) amplify the central rod region of phage T4 gene 37 including the location of the SΔ1 deletion and we used them to screen pseudorevertant phage as well as for sequencing. These primers amplify a 1.4-kb fragment from wild-type T4 DNA but only a 0.36-kb product from T4 37SΔ DNA. Primers recF (GACGAGCTCCTTCGGGTTCCCTTTTTCTTTA, Seq. ID No. 9)) and 37B-2R (TTGGGTAACTCGACATGA, Seq. ID No. 10) amplify a 3.2-kb segment of the tail-fiber gene cluster including the 3′ end of gene 35, gene 36, and the first two-thirds of gene 37. When these primers are used to amplify T4 37SΔ1, a 2.1-kb fragment is produced in which the deletion junction is approximately in the middle. We cloned this 2.1-kb PCR product into pGEM-T (Promega) for sequencing, further modification (see below), and to transfer modified genes into T4 phage by recombination between the plasmid and infecting phage. The construct containing this 2.1-kb insert was designated p37SΔ1.

### C. Recombination of Phage and Plasmid

We transferred modified genes into phage by infecting plasmid bearing cells with T4 37amA481 (whose amber mutation is located in the segment of DNA that is missing in T4 37SΔ1 and its derivatives) and growing the phage to produce a stock. Because MC1061 is not an amber-suppressing strain, only cells where recombination between the plasmid and phage genome occurred would produce viable pseudorevertant phage. We selected recombinant phage from the lysates by plating on BB (su⁰) and screened plaques by PC R to identify which plaques contained the 37SΔ1 deletion.

### D. Measuring Adsorption Rates

Adsorption rates were measured by using a single time point method (Adams, M. H. (1959) Bacteriophages (Interscience, New York)). Briefly, phage were incubated with log phase cells for a fixed time, usually 5 or 10 min at 37° C. (within the phage eclipse period). At that time we diluted the phage/cell mixture into buffer saturated with chloroform to lyse the cells. The number of infectious phage remaining is determined and the adsorption constant is calculated as Kads=(2.3/Ct)log(Po/P), where C is the cell concentration (ml-1), t is the incubation time (in minutes), Po is the infectious phage concentration (ml-1) at time 0, and Pt is the infectious phage concentration (ml⁻¹) at time t.

### E. Construction of SΔ1G5, SΔ1 UCS, SΔ1 ras 1, and SΔ1 ras2

The pentaglycine coding segment in SΔ1G5 was added to the cloned DNA in p37SΔ1/T by using overlapping PCR primers (Sambrook, J. & Russel, D. W. (2001) Molecular Cloning (Cold Spring Harbor Lab. Press, Plainview, N.Y.), 3rd Ed.). Primers 37SΔ1-1F (GGCGATGGTGGCGGCTGGCGGCAATGTACAATTTTACGCTG, Seq. ID No. 11) and 37SΔ1-1R (TACATTGCCGCCACCGCCACCATCGCCATTTAATCTCAA, Seq. ID No. 12) contain complementary sequences corresponding to the Gly-5-containing SΔ1 junction. They were used with the flanking recF and 37B-2R primers to produce two modified half segments that were then recombined using the complementary ends to fuse the two segments and the flanking primers to amplify the whole segment. The entire segment was then cloned into pGEM-T. SΔ1UCS (universal cloning site) was creating by amplifying half segments of the SΔ1 clone with primers 37SΔ1-2F (GGCGATGAGACGGTACCGTCTCAATGTACAATTTTACGCTG, Seq. ID NO. 13) and 37SΔ1-2R (TACATTGAGACGGTACCGTCTCATCGCCATTTAATCTCAA, Seq. ID No. 14). Each primer contains a BsmBI and KpnI site. The two half segments were joined using the KpnI site to create a single segment with two BsmBI sites around the central KpnI site inserted into the SΔ1 junction.

BsmBI cuts at positions 7/11 outside of the recognition site and the two BsmBI sites in p37SΔ1UCS/T are arranged so that the two cuts drop out the center segment (containing both BsmBI sites) leaving the original construct sequence with two different cohesive ends. This arrangement allows for the insertion (with an unambiguous orientation) of any double-stranded oligonucleotide with the correct cohesive ends. Thus, any oligopeptide can be cloned into junction of the SΔ1 deletion. We inserted the ras1 control (nonepitopic for Y13-259); see below) sequence by combining the oligonucleotides SΔ1R-1F (GCGATGGTGGCGGTGGCGCCCCGCGGCGTGGGAAAGAGTGCCCTGACCATCCAGCTG ATCGGTGGCGGTGGCA, Seq. ID No. 15) and SΔ1R-1R (GCATTGCCACCGCCACCGATCAGCTGGATGGTCAGGGCACTCTTTCCCACGCCGCG GGCGCCACCGCCACCA, seq. ID No. 16). Similarly, the ras2 mAb epitope coding D sequence was inserted by using the oligonucleotides SΔ1R-2F (GCGATGGTGGCGGTGGCGAAGAATACTCCGCAATGCGCGACCAGTACATGCGCACC GGTGAAGGTGGCGGTGGCA, Seq. ID No 17) and SΔ1 R-2R (ACATTGCCACCGCCACCTTCACCGGTGCGCATGTACTGGTCGCGCATTGCGCGAGTAT TCTTCGCCACCGCCACCA, Seq. ID No. 18). To anneal each oligonucleotide pail, we mixed the appropriate oligonucleotides in equimolar amounts, boiled the mixtures briefly, and cooled the mixtures slowly to form the appropriate double-strained oligonucleotides with the correct single-stranded extensions. These oligonucleotides were ligated directly into BsmBI digested p37SΔ1UCS/T. The insertions were confirmed by sequencing with the cysF primer.

### F. mAb Inactivation Experiments

We purchased mAb Ab-1 (Y13-259; See Sigal, I. S.; Gibbs, J. B.; D'Alonzo, J. S.; Scolniek, E. M. Proc. Natl. Acad. Sci. USA 1986, 83, 4725-4729) and inactivating peptide from Calbiochem and rabbit anti-rat whole IgG serum from Sigma. The mAb and peptide were resuspended in Dulbeeco's PBS and the anti-serum as used as supplied. For inactivation experiments, we diluted phage to 1010 cells/ml in 10 mM phosphate pH 7.4/10 mM MgSO₄. We added mAb (from a 0.1 mg/ml stock) to 500 μl of diluted phage and incubated the mixture for 30 min (unless otherwise indicated) at room temperature on a rotisserie mixer. Then we added 4 μg of secondary antiserum (from a 2 mg/ml stock) and incubated for 30 min at room temperature.

For the initial experiments shown in FIG. 3A we used 1 μg of mAb, whereas 3 μg or the indicated amount was used for the remaining experiments. For the free epitope inhibition experiment shown in FIG. 3D, we mixed the peptide (EEYSAMRDQVMRTGE, Seq. ID No. 19) and mAb at a 10:1 molar ratio and incubated for 30 min at room temperature. The mAb/peptide mixture was then added to phage as described above.

### G. Electron Microscopy of Phage

Phage and phage/antibody complexes were stained with 1% phosphotungstate (pH 7) on carbon grids. Grids were examined at 100 kV by using a Philips CM10 transmission electron microscope. The final micrograph images were at a magnification of X 73,000.

### Example 7

### Use of Nanosensor of the Invention to Detect Anthrax Spores

In an embodiment the nanosensors of the instant invention contains specific PDLs to a paramagnetic nanoparticle and to a B. anthracis spore (the non-pathgenic Sterne strain). A few good B. anthracis display peptide binding sequences have already been identified. See Brigati, J. et al. “Diagnostic Probes for Bacillus anthracis Spores Selected from a Landscape Phage Library” Clinical Chemistry 2004, 50, 1899-1906. The problem with anthracis spore detection is that there is some background from the closely related B. cercus and B. thuringiensis strains. Our approach can ameliorate this problem by increasing avidity of the composite sensor by adding another one or more PDLs, spaced along each sensor, and thereby binding the strut parallel to the spore surface.

We will first fill two identical capillaries containing sensors with target containing sample (FIG. 2). After the circuit has been “nulled” (by equalizing χ″ between the samples) we will titrate one of the capillaries with free peptide of identical sequence to the PDL, to determine the amount of freed sensor per concentration of added peptide. The null circuit reduces background and is quite sensitive when used to detect phase differences. This is a particularly good method for large targets such as cells or viruses since each nano-sensor can be tightly attached to the target surface (with 2 or 3 binding sites along the rigid strut) and therefore be “clamped” horizontally. The nano-sensor signal should therefore become vanishingly small as each spore builds up a population of “silent” sensors on its surface. After the two identical samples are “nulled” against each other (FIG. 2) one of them will be titrated with free competitive peptide (and the other with buffer). As the number of free sensors buildup, the magnitude of the peak frequency increases at the characteristic χ″ (c)) frequency. Further, by plotting the titration curve for release of sensors as a function of inhibitory free peptide added, the more weakly binding “false positive strains” (such as B. cereus in the ease of anthracis) may well free their bound sensors at lower competitive concentrations, thus providing a two (or more) step curve for χ″(ω) amplitude.

There is another advantage to increasing avidity. For example, Brigati et al. show that the ratio of affinities of their anthracis spore PDLs compared to the B. cereus spore is about 2. If we use a sensor with 3 identical PDLs, theoretically the avidity should rise as the cube if the affinity. Thus if the ratio of avidities for anthracis/cereus should be 8, and thereby facilitate the resolution of the two plateaus of the expected titration—the lower (first plateau) is subtracted from the higher (second plateau) to calculate the concentration of anthracis spores.

As a check for false positive material excess free peptide should give the same null signal as the initial capillary with no target. Multiple attachment of sensors to the large target should enable high sensitivity at low bacterial concentration for high affinity (or avidity) targets. In general, control of solution concentrations of sensors and targets is an important consideration for these assays.

### INCORPORATION BY REFERENCE

All of the U.S. patents and U.S. patent application publications cited herein are hereby incorporated by reference.

1. A sensor for detecting an analyte comprising
(a) a protein rod body portion;
(b) a magnetic particle affixed to the protein rod body portion; and
(c) an analyte interacting moiety disposed on the protein rod body portion at a location remote from the magnetic nanoparticle, wherein the analyte interacting moiety interacts with the analyte to form a sensor product having different hydrodynamic properties than the sensor prior to interaction with the analyte.

2. The sensor of claim 1, wherein the protein rod body portion comprises a tail fiber protein from a T even bacteriophage or a derivative thereof.

3. The sensor of claim 2, wherein the T even bacteriophage is T4 bacteriophage.

4. The sensor of claim 3, wherein the protein rod body portion comprises a gp 34, gp35, or gp 36 tail fiber protein or a derivative thereof.

5. The sensor of claim 4, wherein the protein rod body portion is a fusion protein comprising portions of two or more tail fiber proteins.

6. The sensor of claim 5, wherein the sensor is a multimer of protein rod body portions, each having a magnetic nanoparticle and an analyte binding interacting moiety associated therewith.

7. The sensor of claim 6, wherein the analyte is an antibody, and the analyte interacting moiety is a peptide recognized by the antibody.

8. The sensor of claim 6, wherein the analyte is an antigen, and the analyte interacting moiety comprises a binding fragment of an antibody.

9. The sensor of claim 6, wherein the analyte is a nucleic acid sequence, and the analyte interacting moiety comprises a complementary nucleic acid sequence.

10. The sensor of claim 6, wherein the analyte and the analyte interacting moiety are a hormone and hormone receptor pair.

12. The sensor of claim 6, wherein the analyte and the analyte interacting moiety are an enzyme and enzyme substrate pair.

13. The sensor of claim 6, wherein the analyte and the analyte interacting moiety are an enzyme and enzyme inhibitor pair.

14. The sensor of claim 2, wherein the protein rod body portion is a fusion protein comprising portions of two or more tail fiber proteins.

15. The sensor of claim 3, wherein the sensor is a multimer of protein rod body portions, each having a magnetic nanoparticle and an analyte binding interacting moiety associated therewith.

16. The sensor of claim 15, wherein the analyte is an antibody, and the analyte interacting moiety is a peptide recognized by the antibody.

17. The sensor of claim 15, wherein the analyte is an antigen, and the analyte interacting moiety comprises a binding fragment of an antibody.

18. The sensor of claim 15, wherein the analyte is a nucleic acid sequence, and the analyte interacting moiety comprises a complementary nucleic acid sequence.

19. The sensor of claim 15, wherein the analyte and the analyte interacting moiety are a hormone and hormone receptor pair.

20. The sensor of claim 15, wherein the analyte and the analyte interacting moiety are an enzyme and enzyme substrate pair.

21. The sensor of claim 15, wherein the analyte and the analyte interacting moiety are an enzyme and enzyme inhibitor pair.

22. A method for detecting the presence of an analyte in a liquid sample comprising the steps of:
(a) placing a sensor into the liquid sample, wherein the sensor comprises
(1) a protein rod body portion;
(2) a magnetic particle affixed to the protein rod body portion; and
(3) an analyte interacting moiety disposed on the protein rod body portion at a location remote from the magnetic nanoparticle, wherein the analyte interacting moiety interacts with the analyte to form a sensor product having different hydrodynamic properties than the sensor prior to interaction with the analyte,
(b) applying an AC magnetic field to the sample containing the sensors, and
(c) observing the behavior of the sensor molecules in the magnetic field, wherein a difference in behavior of the sensor from that observed in the absence of analyte is indicative of the presence of analyte in the liquid sample.

23. The method of claim 22, wherein the imaginary component of the magnetic susceptibility of the sensor is observed.

24. The method of claim 22, wherein the imaginary component of the magnetic susceptibility of the sensor is observed using a phase sensitive detector.

25. The method of claim 22, wherein the protein rod body portion of the sensor comprises a tail fiber protein from a T even bacteriophage or a derivative thereof.
